(19) United States
(12) Patent Application Publication
(10) Pub. No.: US 2022/0403018 A1
(43) Pub. Date: Dec. 22, 2022

(54) METHODS OF TREATING LICHEN PLANUS USING INTERLEUKIN (IL-17) ANTAGONISTS

(71) Applicant: NOVARTIS AG, Basel (CH)

(72) Inventors: Deborah Lynn KEEFE, New Rochelle, NY (US); Elisa MUSCIANISI, Atlanta, GA (US); Maximilian REINHARDT, Basel (CH); Xiaoling WEI, Shanghai (CN); Ruquan (Ryan) YOU, Shanghai (CN)

(21) Appl. No.: 17/777,215

(22) PCT Filed: Dec. 4, 2020
(86) PCT No.: PCT/IB2020/061486

§ 371 Date: May 16, 2022

(30) Foreign Application Priority Data
Dec. 6, 2019 (CN) PCT/CN2019/123616

(51) Int. Cl. C07K 16/24; A61P 37/06

(52) U.S. Cl. CPC C07K 16/244; A61P 37/06; A61K 2039/505

(57) ABSTRACT

The present disclosure relates to methods for treating lichen planus (e.g., lichen planopilaris, mucosal lichen planus, cutaneous lichen planus) using Interleukin (IL)-17 antagonists, e.g., secukinumab. Also disclosed herein are IL-17 antagonists, e.g., IL-17 antibodies, such as secukinumab, for treating patients having lichen planus (e.g., lichen planopilaris, mucosal lichen planus, cutaneous lichen planus), as well as medicaments, dosing regimens, pharmaceutical formulations, dosage forms, and kits for use in the disclosed uses and methods.

### TECHNICAL FIELD

The present disclosure relates to methods for treating lichen planus (LP) and lichen planus pilaris (LPP) using IL-17 antagonists, e.g., IL-17 antibodies, e.g., secukinumab.

### BACKGROUND OF THE DISCLOSURE

Lichen planus (LP) is a chronic, inflammatory disorder that can involve the skin, oral or genital mucosa, conjunctiva, and nails (sometimes concomitantly), while maintaining a consistent histologic phenotype. On the skin, (cutaneous lichen planus (CLP)) the disease presents as multiple papules, which can be localized or generalized, that are often extremely itchy and painful. Mucosal disease (mucosal lichen planus (MLP)) can consist of either asymptomatic plaques or extremely painful erosive lesions and ulcers. Lichen planopilaris (LPP), a follicular form of lichen planus, is a rare inflammatory lymphocyte-mediated disorder that selectively involves hair follicles. LPP leads to follicular destruction and, consequently, cicatricial alopecia (Assouly et al. (2009) Semin Cutan Med Surg. 28:3-10). There is broad clinical overlap between the between CLP and MLP subtypes, with many patients presenting overlapping symptoms and lesions. At the same time, each subtype presents at different anatomical regions and with distinct clinical features, e.g., ulceration can be present in the mucosal subtype, but not in the cutaneous subtype, and hair follicle inflammation is a unique feature of lichen planopilaris.

LP affects up to 5% of the worldwide population. Oral or genital involvement occurs in 60-70% of patients, and it may be the sole manifestation of disease in 20-30% of patients. The disease course is unpredictable and typically lasts 1-2 years, but can follow a chronic, relapsing course. Although LP is rather frequently encountered in clinical practice and portends a negative impact on health-related quality-of-life, scarce efforts have been devoted to characterize the management and the proper treatment of LP.

Currently used therapies are mostly symptomatic and employ wide-spectrum immunosuppressive or anti-inflammatory topical (e.g., corticosteroids) and systemic therapies for palliative rather than curative treatment. Systemic immunosuppressants, such as glucocorticoids, cyclosporine, methotrexate, and azathioprine, as well as immunomodulators such as acitretin, help to reduce disease symptoms, but lead to significant side effects following long-term treatment. Furthermore, 30-50% of patients are refractory to current LP therapies, and experience a higher burden of disease due to lack of clinical control, as well as significantly psychological discomfort and social disability, resulting in profoundly impaired quality of life.

Given the severity of LP, along with its chronicity and impact on patients' quality of life, there is a high unmet medical need for safe and effective therapies for the treatment of LP, especially in those patients refractory to standard-of-care systemic and/or topical therapies.

### SUMMARY OF THE DISCLOSURE

Secukinumab (see, e.g., WO2006/013107 and WO2007/117749) is a fully-human monoclonal antibody that selectively neutralizes the human IL-17A cytokine. It has a very high affinity for IL-17, i.e., a K_{D} of about 100-200 pM and an IC₅₀ for in vitro neutralization of the biological activity of about 0.67 nM human IL-17A of about 0.4 nM. Thus, secukinumab inhibits antigen at a molar ratio of about 1:1. This high binding affinity makes secukinumab particularly suitable for therapeutic applications. Furthermore, secukinumab has a long half-life, i.e., about 4 weeks, which allows for prolonged periods between administration, an exceptional property when treating chronic life-long disorders, such as LP.

Several studies confirm that there are increased levels of IL-17A in the serum of LP patients (Pouralibaba, 2013). In addition, biopsies show increased IL-17A produced by the infiltrating lymphocytes. In LPP, an upregulation of the IL-17RA gene is detected in lesional follicles, but not in non-lesion follicles (Hobo, 2018). Nevertheless, these studies do not determine whether IL-17A is a disease driver of LP, or whether IL-17A is merely a disease passenger. In fact, several case reports show lichenoid reactions following treatment with the IL-17 inhibitor, secukinumab, suggesting that IL-17A could provide a protective role in preventing lichenoid reactions (Capusan et al. (2018) JAAD Case Reports 4:521-3 [oral lichenoid eruption following secukinumab treatment]; Doolan et al. (2017) J Clin Exp Dermatol Res 9:1 [cutaneous lichen planus induced by secukinumab]; Komori et al. (2017) J. Derm. 44: e60-e61 [oral lichen planus eruption during secukinumab treatment]; Maglie et al. (2018) Br. J. Derm. 178, 296-308 [oral and cutaneous lichen planus eruption during secukinumab treatment]; Thompson et al. (2016) JAAD Case Reports 2:384-6 [ulcerative, lichenoid mucositis during secukinumab treatment]). In contrast, Solimani et al. (2019) Front. Immunol. 10:1808 describe the efficacy of secukinumab (300 mg s.c. at weeks 0, 1, 2, 3, 4 followed by monthly treatment) in treating three patients with acute and chronic recalcitrant muco-cutaneous LP (i.e., MLP and CLP). However, Solimani et al. (2019) is silent regarding whether secukinumab can be used to treat LPP, a particularly rare form of LP, or whether other dosing regimens of secukinumab can produce desired outcomes while maintaining a favorable risk/benefit profile.

We have now devised novel treatments for LP patients (preferably LP patients who have not adequately responded to a prior treatment with a lichenoid therapy) with IL-17 antagonists, e.g., IL-17 antibodies or antigen-binding fragments thereof, e.g., secukinumab, that are safe, effective and provide sustained responses for patients.

In some embodiments of the disclosed uses, methods and kits, the IL-17 antagonist is an IL-17 antibody or antigen-binding fragment thereof. In some embodiments of the disclosed uses, methods and kits, the IL-17 antibody or antigen-binding fragment thereof is selected from the group consisting of: a) an IL-17 antibody or antigen-binding fragment thereof that binds to an epitope of human IL-17 comprising Leu74, Tyr85, His86, Met87, Asn88, Val124, Thr125, Pro126, Ile127, Val128, His129; b) an IL-17 antibody or antigen-binding fragment thereof that binds to an epitope of human IL-17 comprising Tyr43, Tyr44, Arg46, Ala79, Asp80; c) an IL-17 antibody or antigen-binding fragment thereof that binds to an epitope of an IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 23 to about 35 days; e) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE), and wherein the IL-17 antibody has an in vivo half-life of about 23 to about 30 days; and f) an IL-17 antibody or antigen-binding fragment thereof comprising: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO: 5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO:15; or xi) an immunoglobulin light chain comprising the amino acid sequence set forth as SEQ ID NO:14 and an immunoglobulin heavy chain comprising the amino acid sequence set forth as SEQ ID NO:15.

Disclosed herein are methods of treating lichen planopilaris (LPP), comprising subcutaneously (SC) administering to a patient in need thereof a dose of about 150 mg-about 300 mg of an Interleukin (IL)-17 antibody, or an antigen-binding fragment thereof, weekly during weeks 0, 1, 2, 3, and 4, and every four weeks thereafter, beginning during week 8, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

Disclosed herein are methods of treating of treating lichen planus (LP), comprising subcutaneously (SC) administering to a patient in need thereof a dose of about 150 mg-about 300 mg of an Interleukin (IL)-17 antibody, or an antigen-binding fragment thereof, weekly during weeks 0, 1, 2, 3, and 4, and every two weeks thereafter, beginning during week 6, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

Disclosed herein are methods of treating of treating lichen planus (LP), comprising intravenously (IV) administering to a patient in need thereof a dose of about 4 mg/kg-about 9 mg/kg (preferably about 6 mg/kg) of an Interleukin (IL)-17 antibody, or an antigen-binding fragment thereof, once during week 0, and thereafter administering an IV dose of about 2 mg/kg-about 4 mg/kg (preferably about 3 mg/kg) of the IL-17 antibody, or an antigen-binding fragment thereof every four weeks, beginning during week 4, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

In preferred embodiments, the IL-17 antagonist (e.g., IL-17 antibody or antigen-binding fragment thereof, such as secukinumab) is subcutaneously (SC) administered at a dose of 150 mg or 300 mg. In other preferred embodiments, the IL-17 antagonist (e.g., IL-17 antibody or antigen-binding fragment thereof, such as secukinumab) is intravenously (IV) administered at a dose of 6 mg/kg or 3 mg/kg.

In some embodiments, the IL-17 antagonist (e.g., IL-17 antibody or antigen-binding fragment thereof, such as secukinumab) is administered using an induction regimen, followed by a maintenance regimen. In some embodiments, the induction regimen comprises weekly administration and the maintenance regimen comprises administration every two weeks, every four weeks (monthly), or every eight weeks (every other month). In some embodiments, the induction regimen comprises one administration and the maintenance regimen comprises administration every four weeks (monthly). In some embodiments, the induction regimen comprises every four weeks (monthly) administration and the maintenance regimen comprises administration every eight weeks (every other month).

In some embodiments, the IL-17 antagonist (e.g., IL-17 antibody or antigen-binding fragment thereof, such as secukinumab) is administered SC at a dose of about 300 mg during the induction and maintenance regimen. In some embodiments, the IL-17 antagonist (e.g., IL-17 antibody or antigen-binding fragment thereof, such as secukinumab) is administered SC at a dose of about 150 mg during the induction and maintenance regimen

In some embodiments, the IL-17 antagonist (e.g., IL-17 antibody or antigen-binding fragment thereof, such as secukinumab) is administered IV at a dose of about 6 mg/kg during the induction regimen. In some embodiments, the IL-17 antagonist (e.g., IL-17 antibody or antigen-binding fragment thereof, such as secukinumab) is administered IV at a dose of about 3 mg/kg during the maintenance regimen.

In some embodiments of the disclosed uses, methods and kits, the IL-17 antibody or antigen-binding fragment thereof is a human or humanized antibody. In some embodiments of the disclosed uses, methods and kits, the IL-17 antibody or antigen-binding fragment thereof is secukinumab.

Disclosed herein are methods of treating of treating an adult patient with lichen planopilaris (LPP) that is inadequately controlled with topical corticosteroid therapy or for whom topical corticosteroid therapy is not advisable, comprising administering a dose of about 300 mg secukinumab subcutaneously to said patient during week 0, 1, 2, 3, and 4, and then every four weeks thereafter.

Disclosed herein are methods of treating of treating an adult patient with lichen planus (LP) inadequately controlled with topical corticosteroid therapy or for whom topical corticosteroid therapy is not advisable, comprising administering a dose of about 300 mg secukinumab subcutaneously to said patient during week 0, 1, 2, 3, and 4, and then every two weeks thereafter.

Disclosed herein are methods of treating of treating an adult patient with lichen planus (LP) inadequately controlled with topical corticosteroid therapy or for whom topical corticosteroid therapy is not advisable, comprising, intravenously (IV) administering to the patient a dose of about 6 mg/kg secukinumab once during week 0, and thereafter administering an IV dose of about 3 mg/kg secukinumab every four weeks, beginning during week 4.

### BRIEF DESCRIPTION OF THE FIGURES

FIG. 1 shows the study design of the secukinumab-based LP human clinical trial. Patients enrolled in this trial will have biopsy-confirmed forms of LP (LPP, CLP, or MLP) not adequately controlled with topical therapies. In Treatment Period 1, all patients receive weekly SC injections of blinded study drug (either 300 mg secukinumab or placebo) at weeks 0, 1, 2, 3 and 4, and then every 4 weeks thereafter. In Treatment Period 2, which begins at week 16, patients who were in the placebo treatment arm during Treatment Period 1 will be switched to treatment with SC secukinumab 300 mg every 2 weeks, beginning with an initial, regular induction regimen (five weekly SC injections of 300 mg) followed by maintenance SC secukinumab 300 mg every 2 weeks.

FIG. 2 shows predicted systemic exposure of 300 mg secukinumab using the every 2 week and every 4 weeks dosing intervals during the maintenance regimen.

### DETAILED DESCRIPTION OF THE DISCLOSURE

As used herein, IL-17 refers to interleukin-17A (IL-17A).

The term “comprising” encompasses “including” as well as “consisting,” e.g., a composition “comprising” X may consist exclusively of X or may include something additional, e.g., X+Y.

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +1-10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Exemplary antibodies include secukinumab (Table 1), antibody XAB4 (U.S. Pat. No. 9,193,788), and ixekizumab (U.S. Pat. No. 7,838,638), the disclosures of which are incorporated by reference herein in their entirety.

The term “antigen-binding fragment” of an antibody, as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen-binding fragments include the CDRs of secukinumab as set forth in SEQ ID NOs: 1-6 and 11-13 (Table 1), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IL-17 antibody is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods established in the art. A preferred method for determining the K_{D} of an antibody is by using surface plasmon resonance, or using a biosensor system, e.g., a Biacore® system. In some embodiments, the IL-17 antibody or antigen-binding fragment thereof, e.g., secukinumab, binds human IL-17 with a K_{D} of about 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by assays known in the art, e.g., using a Biacore® analysis.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least about 10% of the measured parameter, by at least 50%, 80% or 90%, and in certain embodiments of the disclosed methods and compositions, the IL-17 antibody used may inhibit greater than 95%, 98% or 99% of IL-17 functional activity.

“Inhibit IL-6” as used herein refers to the ability of an IL-17 antibody or antigen-binding fragment thereof (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6:R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect® (basiliximab) may be conveniently used as a negative control. Supernatant is taken after 16 h stimulation and assayed for IL-6 by ELISA. An IL-17 antibody or antigen-binding fragment thereof, e.g., secukinumab, typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 in human dermal fibroblasts. In some embodiments of the disclosed methods and compositions, IL-17 antibodies or antigen-binding fragments thereof, e.g., secukinumab, and functional derivatives thereof have an IC₅₀ for inhibition of IL-6 production as defined above of about 20 nM or less, more preferably of about 10 nM or less, more preferably of about 5 nM or less, more preferably of about 2 nM or less, more preferably of about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antibody or antigen-binding fragment thereof, e.g., secukinumab, according to the present disclosure, e.g., of a specified sequence (e.g., a variable domain). A “functional derivative” includes a molecule having a qualitative biological activity in common with the disclosed IL-17 antibodies. A functional derivative includes fragments and peptide analogs of an IL-17 antibody as disclosed herein. Fragments comprise regions within the sequence of a polypeptide according to the present disclosure, e.g., of a specified sequence. Functional derivatives of the IL-17 antibodies disclosed herein (e.g., functional derivatives of secukinumab) preferably comprise V_{H} and/or V_{L} domains that have at least about 65%, 75%, 85%, 95%, 96%, 97%, 98%, or even 99% overall sequence identity with the V_{H} and/or V_{L} sequences of the IL-17 antibodies and antigen-binding fragments thereof disclosed herein (e.g., the V_{H} and/or V_{L} sequences of Table 1), and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, the phrase “affected location” refers to any place on a patient's body showing signs of LP, e.g., oral cavity, genitals, conjunctiva, hair, etc.

As used herein, the phrase “active patch” refers to an affected location showing signs of ongoing immune dysregulation, inflammation, swelling, pain, itching, etc.

As used herein, a “therapeutically effective amount” refers to an amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an IL-17 antibody according to the disclosure, for example, secukinumab or ixekizumab, or a pharmaceutical composition comprising said anti-IL-17 antibody, to a subject or to an isolated tissue or cell line from a subject, where the subject has a particular disease (e.g., LP, e.g., cutaneous lichen planus (CLP), mucosal lichen planus (MLP), lichen planopilaris (LPP), or combinations thereof), a symptom associated with the disease (e.g., LP, e.g., CLP, MLP, LPP, or combinations thereof), or a predisposition towards development of the disease (e.g., LP, e.g., CLP, MLP, LPP, or combinations thereof) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, the phrase “population of patients” is used to mean a group of patients. In some embodiments of the disclosed methods, the IL-17 antagonist (e.g., IL-17 antibody, such as secukinumab) is used to treat a population of LP (e.g., CLP, MLP, LPP, or combinations thereof) patients.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient is selected for treatment with the IL-17 antagonist (e.g., IL-17 antibody or antigen-binding fragment thereof, e.g., secukinumab) based on having LP (e.g., CLP, MLP, LPP, or combinations thereof). In some embodiments, the patient is selected for treatment with the IL-17 antagonist (e.g., IL-17 antibody or antigen-binding fragment thereof, e.g., secukinumab) based on having active LP (e.g., CLP, MLP, LPP, or combinations thereof). In some embodiments, the patient is selected for treatment with the IL-17 antagonist (e.g., IL-17 antibody or antigen-binding fragment thereof, e.g., secukinumab) based on having stable cutaneous LP (e.g., CLP, MLP, LPP, or combinations thereof). In some embodiments, the patient is selected for treatment with the IL-17 antagonist (e.g., IL-17 antibody or antigen-binding fragment thereof, e.g., secukinumab) based on having previously had an inadequate response to a prior lichenoid therapy. In some embodiments, the patient is selected for treatment with the IL-17 antagonist (e.g., IL-17 antibody or antigen-binding fragment thereof, e.g., secukinumab) based on being refractory to topical corticosteroid therapy. In some embodiments, the patient is selected for treatment with the IL-17 antagonist (e.g., IL-17 antibody or antigen-binding fragment thereof, e.g., secukinumab) based on having previously had an inadequate response to a topical steroid. In some embodiments, the patient (e.g., patient with lichen planopilaris (LPP)) is selected for treatment with the IL-17 antagonist (e.g., IL-17 antibody or antigen-binding fragment thereof, e.g., secukinumab) based having LP (e.g., LPP) that is inadequately controlled with topical corticosteroid therapy or the patient is one for whom topical corticosteroid therapy is not advisable. Patients for whom “corticosteroid therapy is not advisable” are those, e.g., having allergies to corticosteroid therapy, weakened immune systems, or other co-morbidities and/or co-medications that preclude safe and/or effective treatment with a corticosteroid.

### IL-17 Antagonists

The various disclosed processes, kits, uses and methods utilize an IL-17 antagonist, e.g., IL-17 binding molecule (e.g., soluble IL-17 receptor, IL-17 antibody or antigen-binding fragment thereof, e.g., secukinumab) or IL-17 receptor binding molecule (e.g., IL-17 receptor antibody or antigen-binding fragment thereof). In some embodiments, the IL-17 antagonist is an IL-17 binding molecule, preferably an IL-17 antibody or antigen-binding fragment thereof.

In one embodiment, the IL-17 antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antibody or antigen-binding fragment thereof comprises at least one immunoglobulin light chain variable domain (V_{L′}) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antibody or antigen-binding fragment thereof comprises at least one immunoglobulin heavy chain variable domain (V_{H}) comprising hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13.

In one embodiment, the IL-17 antibody or antigen-binding fragment thereof comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; or ii) hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO: 8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4, SEQ ID NO:5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or h) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

For ease of reference the amino acid sequences of the hypervariable regions of the secukinumab monoclonal antibody, based on the Kabat definition and as determined by the X-ray analysis and using the approach of Chothia and coworkers, is provided in Table 1, below.

TABLE 1
Amino acid sequences of the hypervariable regions of secukinumab.
Light-Chain

| | | |
|---|---|---|
| CDR1′ | Kabat | R-A-S-Q-S-V-S-S-S-Y-L-A (SEQ ID NO: 4) |
| | Chothia | R-A-S-Q-S-V-S-S-S-Y-L-A (SEQ ID NO: 4) |
| CDR2′ | Kabat | G-A-S-S-R-A-T (SEQ ID NO: 5) |
| | Chothia | G-A-S-S-R-A-T (SEQ ID NO: 5) |
| CDR3′ | Kabat | Q-Q-Y-G-S-S-P-C-T (SEQ ID NO: 6) |
| | Chothia | Q-Q-Y-G-S-S-P-C-T (SEQ ID NO: 6) |

Heavy-Chain

| | | |
|---|---|---|
| CDR1 | Kabat | N-Y-W-M-N (SEQ ID NO: 1) |
| CDR1-x | Chothia | G-F-T-F-S-N-Y-W-M-N (SEQ ID NO: 11) |
| CDR2 | Kabat | A-I-N-Q-D-G-S-E-K-Y-Y-V-G-S-V-K-G (SEQ ID NO: 2) |
| CDR2-x | Chothia | A-I-N-Q-D-G-S-E-K-Y-Y (SEQ ID NO: 12) |
| CDR3 | Kabat | D-Y-Y-D-I-L-T-D-Y-Y-I-H-Y-W-Y-F-D-L (SEQ ID NO: 3) |
| CDR3-x | Chothia | C-V-R-D-Y-Y-D-I-L-T-D-Y-Y-I-H-Y-W-Y-F-D-L-W-G |
| | | (SEQ ID NO: 13) |

Secukinumab CDRs according to IMGT are as follows: light chain CDR1 (QSVSSSY; SEQ ID NO:16), CDR 2 (GAS; SEQ ID NO:17), CDR3 (QQYGSSPCT; SEQ ID NO:18); and heavy chain CDR1 (GFTFSNYW; SEQ ID NO:19), CDR2 (INQDGSEK; SEQ ID NO:20), (VRDYYDILTDYYIHYWYFDL; SEQ ID NO:21).

In preferred embodiments, constant region domains also comprise suitable human constant region domains, for instance as described in “Sequences of Proteins of Immunological Interest”, Kabat E. A. et al, US Department of Health and Human Services, Public Health Service, National Institute of Health. The DNA encoding the V_{L} of secukinumab is set forth in SEQ ID NO:9. The DNA encoding the V_{H} of secukinumab is set forth in SEQ ID NO:7.

In some embodiments, the IL-17 antibody or antigen-binding fragment thereof (e.g., secukinumab) comprises the three CDRs of SEQ ID NO:10. In other embodiments, the IL-17 antibody or antigen-binding fragment thereof comprises the three CDRs of SEQ ID NO: 8. In other embodiments, the IL-17 antibody or antigen-binding fragment thereof comprises the three CDRs of SEQ ID NO:10 and the three CDRs of SEQ ID NO:8. CDRs according to Kabat and Chothia of SEQ ID NO:8 and SEQ ID NO:10 may be found in Table 1. CDRs according to IMGT are set forth as SEQ ID NOs:16-18 (light chain CDR1, CDR2, CDR3, respectively) and SEQ ID NOs:19-21 (light chain CDR1, CDR2, CDR3, respectively). The free cysteine in the light chain (CysL97) may be seen, e.g., in SEQ ID NO:6.

In some embodiments, IL-17 antibody or antigen-binding fragment thereof comprises the light chain of SEQ ID NO:14. In other embodiments, the IL-17 antibody or antigen-binding fragment thereof comprises the heavy chain of SEQ ID NO:15. In other embodiments, the IL-17 antibody or antigen-binding fragment thereof comprises the light chain of SEQ ID NO:14 and the heavy domain of SEQ ID NO:15. In some embodiments, the IL-17 antibody or antigen-binding fragment thereof comprises the three CDRs of SEQ ID NO:14. In other embodiments, IL-17 antibody or antigen-binding fragment thereof comprises the three CDRs of SEQ ID NO:15. In other embodiments, the IL-17 antibody or antigen-binding fragment thereof comprises the three CDRs of SEQ ID NO:14 and the three CDRs of SEQ ID NO:15. CDRs of SEQ ID NO:14 and SEQ ID NO:15 may be found in Table 1.

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO: 8) regions. In a similar manner, the light chain framework consists, in sequence, of FR1′ (amino acid 1 to 23 of SEQ ID NO:10), FR2′ (amino acid 36 to 50 of SEQ ID NO:10), FR3′ (amino acid 58 to 89 of SEQ ID NO:10) and FR4′ (amino acid 99 to 109 of SEQ ID NO:10) regions.

In one embodiment, the IL-17 antibody or antigen-binding fragment thereof (e.g., secukinumab) is selected from a human IL-17 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1′, CDR2′, and CDR3′ and the constant part or fragment thereof of a human light chain, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antibody or antigen-binding fragment thereof as used in the disclosed methods may comprise a derivative of the IL-17 antibodies set forth herein by sequence (e.g., pegylated variants, glycosylation variants, affinity-maturation variants, etc.). Alternatively, the V_{H} or V_{L} domain of an IL-17 antibody or antigen-binding fragment thereof used in the disclosed methods may have V_{H} or V_{L} domains that are substantially identical to the V_{H} or V_{L} domains set forth herein (e.g., those set forth in SEQ ID NO:8 and 10). A human IL-17 antibody disclosed herein may comprise a heavy chain that is substantially identical to that set forth as SEQ ID NO:15 and/or a light chain that is substantially identical to that set forth as SEQ ID NO:14. A human IL-17 antibody disclosed herein may comprise a heavy chain that comprises SEQ ID NO:15 and a light chain that comprises SEQ ID NO:14. A human IL-17 antibody disclosed herein may comprise: a) one heavy chain which comprises a variable domain having an amino acid sequence substantially identical to that shown in SEQ ID NO:8 and the constant part of a human heavy chain; and b) one light chain which comprises a variable domain having an amino acid sequence substantially identical to that shown in SEQ ID NO:10 and the constant part of a human light chain.

Alternatively, an IL-17 antibody or antigen-binding fragment thereof used in the disclosed methods may be an amino acid sequence variant of the reference IL-17 antibodies set forth herein, as long as it contains CysL97. The disclosure also includes IL-17 antibodies or antigen-binding fragments thereof (e.g., secukinumab) in which one or more of the amino acid residues of the V_{H} or V_{L} domain of secukinumab (but not CysL97), typically only a few (e.g., 1-10), are changed; for instance by mutation, e.g., site directed mutagenesis of the corresponding DNA sequences. In all such cases of derivative and variants, the IL-17 antibody or antigen-binding fragment thereof is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts as described in Example 1 of WO 2006/013107.

In some embodiments, the IL-17 antibodies or antigen-binding fragments thereof, e.g., secukinumab, bind to an epitope of mature human IL-17 comprising Leu74, Tyr85, His86, Met87, Asn88, Val124, Thr125, Pro126, Ile127, Val128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain. The residue numbering scheme used to define these epitopes is based on residue one being the first amino acid of the mature protein (i.e., IL-17A lacking the 23 amino acid N-terminal signal peptide and beginning with glycine). The sequence for immature IL-17A is set forth in the Swiss-Prot entry Q16552. In some embodiments, the IL-17 antibody has a K_{D} of about 100-200 pM (e.g., as determined by a Biacore® assay). In some embodiments, the IL-17 antibody has an IC₅₀ of about 0.4 nM for in vitro neutralization of the biological activity of about 0.67 nM human IL-17A. In some embodiments, the absolute bioavailability of subcutaneously (SC) administered IL-17 antibody has a range of about 60%-about 80%, e.g., about 76%. In some embodiments, the IL-17 antibody, such as secukinumab, has an elimination half-life of about 4 weeks (e.g., about 23 to about 35 days, about 23 to about 30 days, e.g., about 30 days). In some embodiments, the IL-17 antibody (such as secukinumab) has a Tₘₐₓ of about 7-8 days.

Particularly preferred IL-17 antibodies or antigen-binding fragments thereof used in the disclosed methods are human antibodies, especially secukinumab as described in Examples 1 and 2 of WO 2006/013107. Other preferred IL-17 antibodies for use in the disclosed methods, kits and regimens are those set forth in U.S. Pat. Nos. 8,057,794; 8,003,099; 8,110,191; and 7,838,638 and US Published Patent Application Nos: 20120034656 and 20110027290, which are incorporated by reference herein in their entirety.

### Methods of Treatment and Uses of IL-17 Antagonists

The disclosed IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof), may be used in vitro, ex vivo, or incorporated into pharmaceutical compositions and administered in vivo to treat patients having LP (e.g., CLP, MLP, LPP, or combinations thereof) (e.g., human patients).

Lichen planus (LP) is an inflammatory autoimmune disease that affects both cutaneous and mucosal skin. Although pathophysiology is not yet fully defined, LP is a T-cell mediated disorder that demonstrates an increased Th1 cytokine expression as well as T-cell reactivity against basement membrane zone components. There are several clinical types of LP that share similar histopathological features: cutaneous lichen planus (CLP) (LP occurring on cutaneous skin), mucosal lichen planus (MLP) (LP occurring on mucosal skin), lichen planopilaris (LPP) (LP affecting hair follicles), lichen planus of the nails, lichen planus pigmentosus, lichenoid drug eruption. Immunological and histopathological features of LP are described in, e.g., Arora et al. (2014) Indian J Dermatol. 59(3): 257-261, and Atas et al. (2016) Postepy Dermatol Alergol. 33(3):188-92.

LP is typically confirmed by biopsy, meaning the disorder has been “biopsy-confirmed”. In preferred embodiments, the patient has biopsy-confirmed LP (MLP, CLP, LPP, or combinations thereof).

In preferred embodiments, the LP patient to be treated using the disclosed methods, uses, kits, etc. has LPP. In preferred embodiments, the LP patient to be treated using the disclosed methods, uses, kits, etc. has MLP. In preferred embodiments, the LP patient to be treated using the disclosed methods, uses, kits, etc. has CLP.

As used herein, the phrase “stable cutaneous LP” refers to LP wherein the patient: 1) has a baseline Investigator's Global Assessment (IGA) of ≥3; and 2) is refractory to topical corticosteroid therapy or has had inadequate response to topical steroids. In some embodiments, the LP patient to be treated using the disclosed methods, uses, kits, etc. has stable cutaneous LP.

As used herein, the phrases “inadequately controlled”, “inadequate response”, “did not adequately respond” and the like refer to treatments that produce an insufficient response or treatment failure in a patient, e.g., following treatment with a given agent a patient still has one or more pathological symptoms of the disorder, e.g., in the case of LP, symptoms include itching, patches of hair loss, pain, burning, lesions, etc. In some embodiments, prior to administering the IL-17 antagonist, the patient has had an inadequate response to prior treatment with a lichenoid therapy. In some embodiments, the patient has had an inadequate response to prior treatment with a topical steroid (e.g., a topical corticosteroid).

A patient who has responded adequately to treatment with a lichenoid therapy (e.g., a corticosteroid), but has discontinued due to a side effect is termed “intolerant”. In some embodiments, the patient having LP (e.g., LPP, MLP, CLP, or combinations thereof) to be treated using the disclosed methods, uses, kits, etc. is intolerant to a prior lichenoid therapy (e.g., a corticosteroid). In some embodiments, the patient is intolerant to topical corticosteroid therapy, e.g., high potency corticosteroids (according to the WHO definition).

Refractory refers to a particular type of inadequate response, i.e., by “refractory” is meant that the patient has been treated with at least 4 weeks of high potency lichenoid therapy (e.g., a corticosteroid) without significant improvement. In some embodiments, the patient having LP (e.g., LPP, MLP, CLP, or combinations thereof) who is treated according to the disclosed methods, uses, kits, etc. is refractory to treatment with a prior lichenoid therapy. In some embodiments, the patient is refractory to topical corticosteroid therapy, e.g., high potency corticosteroids (according to the WHO definition).

As used herein, “lichenoid therapy” refers to LP treatments employing LP agents (e.g., small molecules, biological therapies, creams, ointments, etc.) or employing an LP modality (e.g., phototherapy), including topical therapies, systemic therapies, phototherapies, retinoids, and combinations thereof. These include topical therapies in the form of creams, lotions, sprays, or shampoos (e.g., low-medium potency corticosteroids [Group IV-VII according to WHO guidelines, see Bolognia J L, Jorizzo J L, Schaffer J V. Glucocorticosteroids. Dermatology. 3rd ed. 2012. Ch 125, 2075-88; Ference J D, Last A R. Choosing topical corticosteroids. Am Fam Physician. 2009 Jan. 15; 79(2):135-40]); over the counter (OTC) emollients, shampoos and lubricants for the treatment of itch and/or pain, e.g. anti-itch lotions containing menthol, pramoxine or anti-histamines; mixed medication for oral pain (for example, OTC mouthwashes containing diphenhydramine, viscous lidocaine, antacid, nystatin or corticosteroids); local anesthetics, systemic agents (e.g., biological agents, e.g., TNF alpha inhibitors, such as adalimumab, infliximab, certolizumab and etanercept, alefacept, briakinumab, efalizumab, ustekinumab, ixekizumab, brodalumab, guselkumab, risankizumab, tildrakizumab, non-biological immunomodulating treatments, e.g., methotrexate, apremilast, systemic corticosteroids, cyclosporine, cyclophosphamide, sulphasalazine, azathioprin, mycophenolate mofetil, dapson, hydroxychloroquine); retinoids (e.g., alitretinoin); intralesional corticosteroid injections; phototherapy (e.g. UVB). photochemotherapy (e.g. psoralen and UVA (PUVA)); topical calcineurin inhibitors (cyclosporine, tacrolimus, pimecrolimus) or topical Vitamin D analogues; topical corticosteroids of high—ultrahigh potency (Group I, II, III as per WHO definition); anti-fungal drugs with known anti-inflammatory properties, e.g., griseofulvin, itraconazole, Betamethasone, dexamethasone, INCB018424, triamcinolone, apremilast, turmeric past, glucosamine sulfate, triamcinolone acetonide, sesame oil, betamethasone dipropionate, clobetasol propionate, probiotics (e.g., Bifidobacterium animalis subst. lactis HN019, lactobacilli reuteri), omega-3, prednisone, prednisolone, platelet rich plasma, orabase paste, lycopene, topical chamomile, green tea, CO2 laser treatment, polybiotics, photobiomodulation, metronidazole, doxycycline, minocycline, cedar honey, purslane, curcuminoids, alefacept, hexaminolevulinate, hydroxychloroquine, adcortyl, efalizumab, fluocinolone, co-enzyme Q10 mucoadhesive tablets, Chamaemelum nobile, sirolimus, tacrolimus, qingxuan decoction, NSAID topical rinse, NSAIDs, quercetin, NAVS naphthalan, valchlor, bupivacaine, oatmeal baths. Further information on lichenoid therapies (including LP agents) may be found in Husein-ElAhmed (2019) J Eur Acad Dermatol Venereol. doi: 10.1111/jdv.15771, which is incorporated by reference herein in its entirety.

Preferred low-medium potency topical corticosteroids are Desoximetasone Cream, 0.05%, Fluocinolone acetonide Ointment, 0.025%, Fludroxycortide Ointment, 0.05%, Hydrocortisone valerate Ointment, 0.2%, Triamcinolone acetonide Cream, 0.1%, Betamethasone dipropionate Lotion, 0.02%, Betamethasone valerate Cream, 0.1%, Fluocinolone acetonide Cream, 0.025%, Fludroxycortide Cream, 0.05%, Hydrocortisone butyrate Cream, 0.1%, Hydrocortisone valerate Cream, 0.2%, Triamcinolone acetonide Lotion, 0.1%, Betamethasone valerate Lotion, 0.05%, Desonide Cream, 0.05%, Fluocinolone acetonide Solution, 0.01%, Dexamethasone sodium phosphate Cream, 0.1%, Hydrocortisone acetate Cream, 1%, Methylprednisolone acetate Cream, 0.25%.

As used herein, “induction” refers to the portion of a therapy that induces lowering or remission of disease burden. Thereafter, a patient is treated with a “maintenance” regimen to maintain the patient in a disease-free (or relapse-free) state.

The effectiveness of an LP treatment may be assessed using various known methods and tools that measure lichenoid disease. Such tests include, e.g., biopsy and subsequent histopatholoy, Physician Assessment of Surface Area of Disease (PSAD) (see NCT00285779), Investigator's Global Assessment (IGA) score (where a 0/1 score indicates clearance or almost clearance of symptoms, and a score of ≥3 represents moderate to severe disease); the Reticular erythematous Ulcerative (REU) score (Piboonniyom et al (2005) Oral Surg Oral Med Oral Pathol Oral Radiol Endod; 99(6):696-703); Dermatology Life Quality Index (DLQI) (see, e.g., Finlay (1994) Clin Exp Dermatol. 19(3):210-6, where a 0/1 score indicates that the disease has no effect on a patient's quality of life); Body Surface Area (BSA) (whereby a health care professional estimates the percentage of a patient's body surface area that is affected by LP; patient obtaining a BSA below 1% is considered a responder to treatment according to the disclosed methods, kits and uses); Peak Pruritus Numerical Rating Scale (NRS) (see, e.g., Yosipovitch et al. (2019) Br J Dermatol. 181(4):761-769); LPP Activity Index (LPPAI) (see, e.g., Chiang et al, (2010) J Am Acad Dermatol 62:387-92); Oral Lichen Planus Symptom Severity Measure (OLPSSM) (see, e.g., Burke (2019) Oral Dis. September; 25(6):1564-1572); SCALPDEX (see, e.g., Chen (2002) Arch. Dermatol. 138:803-07); Patient Assessment of Overall Disease Severity; Lichen Planus Symptoms Inventory; Epworth Sleepiness Scale (ESS); pain using Visual Analogue Scale (VAS) (see, e.g., Chainani-Wu et al. (2008) Oral Surg Oral Med Oral Pathol Oral Radiol Endod. 105(1):51-8; Chaudhary S. (2004) Aust Dent J.; 49(4):192-195); Trichoscopy score; Modified Oral Mucositis Index (MOMI) (see, e.g., Chainani-Wu et al. (2008), supra), Oral Health Impact Profile (OHIP-14) (see, e.g., Mary et al. (2017) J Clin Diagn Res. 11(8): ZC78-ZC81), and a patient's ability to discontinue or reduce topical treatments.

We have generated a unique Investigator's Global Assessment (IGA) score that may be used to assess LP severity (and LP patient's response to treatment) for MLP, CLP and LPP. This IGA (Table 2) provides a harmonized, 5-point grading system to assess disease severity for patients of all three LP subtypes (MLP, CLP and LPP). The predominant subtype defines the IGA score of the patient. The IGA grading is based on the predominant subtype alone. In addition, the IGA score is also collected separately for concomitant subtypes, if present

TABLE 2
Investigator's Global Assessment (IGA) for Lichen Planus. The grading should be
mainly driven by the lesion characteristics. Symptoms, such as pain or pruritus, may or may not
be associated.

| | Cutaneous Lichen | Mucosal Lichen | |
|---|---|---|---|
| IGA score | Planus | Planus | Lichen Planopilaris |
| 0—Clear | No disease. Possible | No disease. | No active disease. |
| | flat, hyperpigmented | | Hypopigmented, |
| | lesions. | | cicatricial patches. |
| | | | Pull test negative. |
| 1—Minimal | Barely palpable (<0.5 | Reticular, white, | 1 or 2 hairless |
| | mm), scattered | patch-type striations | patches, |
| | papules, mild | involving any oral or | predominantly |
| | erythema. | genital mucosal site(s) | inactive disease |
| | Typically associated | (“Wickham Striae”). | (hypopigmented |
| | with minimal | Absence of ulcers. | cicatricial patches). |
| | pruritus. | Typically associated | Absence of disease |
| | | with no symptoms. | spreading. Pull test |
| | | | negative. Minimal |
| | | | scalp erythema and/or |
| | | | perifollicular |
| | | | erythema without |
| | | | scale. |
| | | | Typically associated |
| | | | with minimal |
| | | | pruritus. |
| 2—Mild | Moderately elevated | Reticular, plaque- | 2-4 hairless patches |
| | papules (<1 mm) | type, mucosal lichen | with perifollicular |
| | and/or small plaques | planus involving any | erythema, rare |
| | (<2 cm²). Involvement | oral or genital | perifollicular |
| | of limited areas, | mucosal site(s). | hyperkeratosis and |
| | erythema. | Absence of or | interfollicular scale. |
| | Typically associated | minimal ulcers. | Stable or slowly |
| | with mild pruritus. | Typically associated | spreading disease. |
| | | with mild pain or | Pull test negative. |
| | | sensitivity. | Typically associated |
| | | | with mild pruritus. |
| 3—Moderate | Thick, elevated (<2 | Presence of unilateral | 5 or more hairless |
| | mm), hypertrophic, | ulcers involving any | patches with |
| | violaceous papules or | oral or genital | perifollicular and |
| | presence of non- | mucosal site(s). | interfollicular |
| | hypertrophic, plaque- | Possible desquamative | erythema, follicular |
| | like disease (>2 cm²), | gingivitis or vulvitis. | hyperkeratosis and |
| | either generalized or | Typically associated | interfollicular scaling. |
| | involving specific | with moderate pain | Active spreading |
| | areas or specific | and sensitivity. | disease. Pull test |
| | locations (e.g. face, | | positive for telogen |
| | neck). Red/violaceous | | hairs. |
| | erythema, possible | | Typically associated |
| | scales. | | with moderate |
| | Typically associated | | pruritus, pain and/or |
| | with moderate | | burning. |
| | pruritus. | | |
| 4—Severe | Generalized, papular | Presence of bilateral | 5 or more hairless |
| | or plaque-type (>2 | or extensive unilateral | patches with |
| | cm²), hypertrophic or | ulcers involving any | perifollicular and |
| | non-hypertrophic, | oral or genital | interfollicular, intense |
| | elevated lesions (>2 | mucosal site(s). | erythema, extensive |
| | mm). Marked | Possible esophageal | perifollicular and |
| | red/violaceous | involvement | interfollicular scaling. |
| | erythema. Possible | (established by | Crusting, pustules. |
| | scales, blisters and | endoscopy). | Active spreading |
| | ulcers. | Typically associated | disease. Pull test |
| | Typically associated | with severe pain and | positive for telogen or |
| | with severe pruritus. | sensitivity. | anagen hairs. |
| | | | Typically associated |
| | | | with severe pruritus, |
| | | | pain and/or burning. |

As used herein, the term “baseline” and the like (e.g., “baseline value”) refer to the value of a given variable prior to a patient being treated with the IL-17 antibody or antigen-binding fragment thereof.

In some embodiments, the patient is an adult human patient having LP (e.g., LPP, MLP, CLP, or combinations thereof). Is some embodiments, the patient is a pediatric human patient having LP (e.g., LPP, MLP, CLP, or combinations thereof). The upper age limit used to define a pediatric patient varies among experts, and can include adolescents up to the age of 21 (see, e.g., Berhman R E, Kliegman R, Arvin A M, Nelson W E. Nelson Textbook of Pediatrics, 15th Ed. Philadelphia: W.B. Saunders Company; 1996; 2. Rudolph A M, et al. Rudolph's Pediatrics, 21st Ed. New York: McGraw-Hill; 2002; and Avery M D, First L R. Pediatric Medicine, 2nd Ed. Baltimore: Williams & Wilkins; 1994). As used herein, the term “Pediatric” generally refers to a human who is sixteen years old or younger, which is the definition of a pediatric human used by the US FDA.

In some embodiments, the pediatric patient is administered a SC dose of the IL-17 antibody (e.g., secukinumab) weekly during week 0, 1, 2, 3, and 4, and then every two weeks or four weeks thereafter as a dose of about 150 mg-about 300 mg (e.g., 150 mg or 300 mg), regardless of the patient's weight.

In some embodiments, the pediatric patient is administered a SC dose of the IL-17 antibody (e.g., secukinumab) weekly during week 0, 1, 2, 3, and 4, and then every two weeks or every four weeks thereafter as a dose of about 75 mg if the patient weighs <25 kg or about 150 mg if the patient weighs >25 kg. In some embodiments, the pediatric patient is administered a SC dose of the IL-17 antibody (e.g., secukinumab) weekly during week 0, 1, 2, 3, and 4, and then every two weeks or every four weeks thereafter as a dose of about 75 mg if the patient weighs <50 kg or about 150 mg if the patient weighs >50 kg.

In some embodiments, the pediatric patient is administered a SC dose of the IL-17 antibody (e.g., secukinumab) weekly during week 0, 1, 2, 3, and 4, and then every two weeks or every four weeks thereafter as a dose of about 150 mg if the patient weighs <25 kg or 300 mg if the patient weighs >25 kg. In some embodiments, the pediatric patient is administered a SC dose of the IL-17 antibody (e.g., secukinumab) weekly during week 0, 1, 2, 3, and 4, and then every two weeks or every four weeks thereafter as a dose of about 150 mg if the patient weighs <50 kg or 300 mg if the patient weighs >50 kg.

In some embodiments, the pediatric patient is administered an IV dose of the IL-17 antibody (e.g., secukinumab) of about 4 mg/kg-about 9 mg/kg (preferably about 6 mg/kg) once during week 0, and thereafter, as an IV dose of about 2 mg/kg-about 4 mg/kg (preferably about 3 mg/kg) every 4 weeks (monthly), beginning during week 4.

The IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof). In preferred embodiments, the pharmaceutical compositions for use in the disclosed methods comprise secukinumab at 150 mg/ml.

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise ready-to-use liquid formulations.

Antibodies, e.g., antibodies to IL-17, are typically formulated either in ready-to-use aqueous forms for parenteral administration or as lyophilisates for reconstitution with a suitable diluent prior to administration. In preferred embodiments of the disclosed methods and uses, the IL-17 antagonist, e.g., IL-17 antibody, e.g., secukinumab, is formulated as ready-to-use (i.e., a stable ready-to-use) liquid pharmaceutical formulation. In some embodiments of the disclosed methods and uses, the IL-17 antagonist, e.g., IL-17 antibody, e.g., secukinumab, is formulated as a lyophilisate. Suitable lyophilisate formulations can be reconstituted in a small liquid volume (e.g., 2 mL or less, e.g., 2 mL, 1 mL, etc.) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are known in the art. When a therapeutically effective amount of an IL-17 antagonist, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's solution, dextrose, dextrose and sodium chloride, lactated Ringer's solution, or other vehicle as known in the art.

In practicing some of the methods of treatment or uses of the present disclosure, a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) is administered to a patient, e.g., a mammal (e.g., a human). While it is understood that the disclosed methods provide for treatment of LP (e.g., CLP, LPP, MLP, or combinations thereof) patients using an IL-17 antagonist (e.g., secukinumab), this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered in accordance with the methods of the disclosure either alone or in combination with other agents and therapies for treating LP (e.g., CLP, LPP, MLP, or combinations thereof) patients, e.g., in combination with at least one additional LP agent or lichenoid therapy. When co-administered with one or more additional LP agent or lichenoid therapy, an IL-17 antagonist may be administered either simultaneously with the other agent, or sequentially. If administered sequentially, the attending health care professional will decide on the appropriate sequence of administering the IL-17 antagonist in combination with other agents and the appropriate dosages for co-delivery.

Various lichenoid therapies may be beneficially combined with the disclosed IL-17 antibodies, such as secukinumab, during treatment of LP (e.g., CLP, LLP, MLP, or combinations thereof). Non-limiting examples include LP agents (e.g., small molecules, biological therapies, creams, ointments, etc.) and LP modalities (e.g., phototherapy), such as topical therapies, systemic therapies, phototherapies, retinoids, and combinations thereof. These include topical therapies in the form of creams, lotions, sprays, or shampoos (e.g., low-medium potency corticosteroids [Group IV-VII according to WHO guidelines]); over the counter (OTC) emollients, shampoos and lubricants for the treatment of itch and/or pain, e.g. anti-itch lotions containing menthol, pramoxine or anti-histamines; mixed medication for oral pain (for example, OTC mouthwashes containing diphenhydramine, viscous lidocaine, antacid, nystatin or corticosteroids); local anesthetics, systemic agents (e.g., biological agents, e.g., TNF alpha inhibitors, such as adalimumab, infliximab, certolizumab and etanercept, alefacept, briakinumab, efalizumab, ustekinumab, ixekizumab, brodalumab, guselkumab, risankizumab, tildrakizumab, non-biological immunomodulating treatments, e.g., methotrexate, apremilast, systemic corticosteroids, cyclosporine, cyclophosphamide, sulphasalazine, azathioprin, mycophenolate mofetil, dapson, hydroxychloroquine); retinoids (e.g., alitretinoin); intralesional corticosteroid injections; phototherapy (e.g. UVB). photochemotherapy (e.g. psoralen and UVA (PUVA)); topical calcineurin inhibitors (cyclosporine, tacrolimus, pimecrolimus) or topical Vitamin D analogues; topical corticosteroids of high—ultrahigh potency (Group I, II, III as per WHO definition); anti-fungal drugs with known anti-inflammatory properties, e.g., griseofulvin, itraconazole, Betamethasone, dexamethasone, INCB018424, triamcinolone, apremilast, turmeric past, glucosamine sulfate, triamcinolone acetonide, sesame oil, betamethasone dipropionate, clobetasol propionate, probiotics (e.g., Bifidobacterium animalis subst. lactis HN019, lactobacilli reuteri), omega-3, prednisone, prednisolone, platelet rich plasma, orabase paste, lycopene, topical chamomile, green tea, CO2 laser treatment, polybiotics, photobiomodulation, metronidazole, doxycycline, minocycline, cedar honey, purslane, curcuminoids, alefacept, hexaminolevulinate, hydroxychloroquine, adcortyl, efalizumab, fluocinolone, co-enzyme Q10 mucoadhesive tablets, Chamaemelum nobile, sirolimus, tacrolimus, qingxuan decoction, NSAID topical rinse, NSAIDs, quercetin, NAVS naphthalan, valchlor, bupivacaine, and combinations thereof.

Preferred LP agents for use in the disclosed kits and methods in combination with the IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) are over-the-counter emollients and lubricants for pain (including anti-histamines), magic mouthwash, nystatin oral, and low-medium potency corticosteroids (Group IV-VII as per WHO definition) (e.g., Desoximetasone Cream, 0.05%, Fluocinolone acetonide Ointment, 0.025%, Fludroxycortide Ointment, 0.05%, Hydrocortisone valerate Ointment, 0.2%, Triamcinolone acetonide Cream, 0.1%, Betamethasone dipropionate Lotion, 0.02%, Betamethasone valerate Cream, 0.1%, Fluocinolone acetonide Cream, 0.025%, Fludroxycortide Cream, 0.05%, Hydrocortisone butyrate Cream, 0.1%, Hydrocortisone valerate Cream, 0.2%, Triamcinolone acetonide Lotion, 0.1%, Betamethasone valerate Lotion, 0.05%, Desonide Cream, 0.05%, Fluocinolone acetonide Solution, 0.01%, Dexamethasone sodium phosphate Cream, 0.1%, Hydrocortisone acetate Cream, 1%, Methylprednisolone acetate Cream, 0.25%, and combinations thereof).

A skilled artisan will be able to discern the appropriate dosages of the above LP agents for co-delivery with the disclosed IL-17 antibodies, such as secukinumab.

An IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) is conveniently administered parenterally, e.g., intravenously (e.g., into the antecubital or other peripheral vein), intramuscularly, or subcutaneously. The duration of intravenous (IV) therapy using a pharmaceutical composition of the present disclosure will vary, depending on the severity of the disease being treated and the condition and personal response of each individual patient. Also contemplated is subcutaneous (SC) therapy using a pharmaceutical composition of the present disclosure. The health care provider will decide on the appropriate duration of IV or SC therapy and the timing of administration of the therapy, using the pharmaceutical composition of the present disclosure. In preferred embodiments, the IL-17 antagonist (e.g., secukinumab) is administered via the subcutaneous (SC) route.

The IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient (e.g., a patient having LPP) SC, e.g., at about 150 mg-about 300 mg (e.g., about 150 mg, about 300 mg) weekly during weeks 0, 1, 2, 3, and 4, and thereafter administered to the patient SC, e.g., at about 150 mg-about 300 mg (e.g., about 150 mg, about 300 mg) monthly (every 4 weeks), beginning during week 8. In this manner, the patient is dosed SC with about 150 mg-about 300 mg (e.g., about 150 mg or about 300 mg) of the IL-17 antagonist (e.g., secukinumab) during weeks 0, 1, 2, 3, 4, 8, 12, 16, 20, etc.

The IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered SC to the patient (e.g., a patient having LPP, MLP, CLP or combinations thereof), e.g., at about 150 mg-about 300 mg (e.g., about 150 mg, about 300 mg) weekly during weeks 0, 1, 2, 3, and 4, and thereafter administered to the patient SC, e.g., at about 150 mg-about 300 mg (e.g., about 150 mg, about 300 mg) every 2 weeks, beginning during week 6. In this manner, the patient is dosed SC with about 150 mg-about 300 mg (e.g., about 150 mg or about 300 mg) of the IL-17 antagonist (e.g., secukinumab) during weeks 0, 1, 2, 3, 4, 6, 8, 10, 12, 14, 16, 18, 20, etc.

Alternatively, the IL-17 antagonist (e.g., IL-17 antibody or antigen-binding fragment thereof, such as secukinumab) may be administered intravenously (IV) to the patient (e.g., a patient having LPP, MLP, CLP or combinations thereof). Preferred IV regimens (dose and administration scheme) for use with the disclosed IL-17 antagonists to treat LP are provided in

TABLE 3
Preferred IV/IV regimens for use in the disclosed methods employing an IL-17
antagonist, e.g., IL-17 binding molecule (e.g., IL-17 antibody or antigen-binding fragment
thereof, e.g., secukinumab) or IL-17 receptor binding molecule (e.g., IL-17 receptor antibody or
antigen-binding fragment thereof).

| Loading regimen (IV) | Maintenance regimen (IV) |
|---|---|
| about 4.0 mg/kg (e.g., 4.0 mg/kg) once during | about 2.0 mg/kg (e.g., 2.0 mg/kg) monthly |
| week 0 | (every 4 weeks), beginning during week 4 |
| about 4.0 mg/kg (e.g., 4.0 mg/kg) once during | about 3.0 mg/kg (e.g., 3.0 mg/kg) monthly |
| week 0 | (every 4 weeks), beginning during week 4 |
| about 4.0 mg/kg (e.g., 4.0 mg/kg) once during | about 4.0 mg/kg (e.g., 4.0 mg/kg) monthly |
| week 0 | (every 4 weeks), beginning during week 4 |
| about 5.0 mg/kg (e.g., 5.0 mg/kg) once during | about 2.5 mg/kg (e.g., 2.5 mg/kg) monthly |
| week 0 | (every 4 weeks), beginning during week 4 |
| about 6.0 mg/kg (e.g., 6.0 mg/kg) once during | about 2.0 mg/kg (e.g., 2.0 mg/kg) monthly |
| week 0 | (every 4 weeks), beginning during week 4 |
| about 6.0 mg/kg (e.g., 6.0 mg/kg) once during | about 3.0 mg/kg (e.g., 3.0 mg/kg) monthly |
| week 0 | (every 4 weeks), beginning during week 4 |
| about 6.0 mg/kg (e.g., 6.0 mg/kg) once during | about 4.0 mg/kg (e.g., 4.0 mg/kg) monthly |
| week 0 | (every 4 weeks), beginning during week 4 |
| about 7.0 mg/kg (e.g., 7.0 mg/kg) once during | about 3.5 mg/kg (e.g., 3.5 mg/kg) monthly |
| week 0 | (every 4 weeks), beginning during week 4 |
| about 8.0 mg/kg (e.g., 8.0 mg/kg) once during | about 4.0 mg/kg (e.g., 4.0 mg/kg) monthly |
| week 0 | (every 4 weeks), beginning during week 4 |
| about 9.0 mg/kg (e.g., 9.0 mg/kg) once during | about 2.0 mg/kg (e.g., 2.0 mg/kg) monthly |
| week 0 | (every 4 weeks), beginning during week 4 |
| about 9.0 mg/kg (e.g., 9.0 mg/kg) once during | about 3.0 mg/kg (e.g., 3.0 mg/kg) monthly |
| week 0 | (every 4 weeks), beginning during week 4 |
| about 9.0 mg/kg (e.g., 9.0 mg/kg) once during | about 4.0 mg/kg (e.g., 4.0 mg/kg) monthly |
| week 0 | (every 4 weeks), beginning during week 4 |
| about 10 mg/kg (e.g., 10 mg/kg) monthly | about 10 mg/kg (e.g., 10 mg/kg) every two |
| (every 4 weeks) during week 0, 4, 8 | months (every 8 weeks), beginning during |
| | week 16 |

In some embodiments, it is contemplated that the IL-17 antagonist (e.g., IL-17 antibody or antigen-binding fragment thereof, such as secukinumab) may be IV administered to the patient (e.g., a patient having LPP, MLP, CLP or combinations thereof) at a dose of about 4 mg/kg-about 9 mg/kg (preferably about 6 mg/kg) once during week 0, and thereafter, as an IV dose of about 2-about 4 mg/kg (preferably about 3 mg/kg) every 4 weeks (monthly), beginning during week 4. In this manner, the patient is dosed IV with about 4 mg/kg-about 9 mg/kg (e.g., about 6 mg/kg) of the IL-17 antagonist (e.g., secukinumab) during weeks 0, 4, 8, 12, 16, 20, etc. In a preferred embodiment, the IL-17 antagonist (e.g., IL-17 antibody or antigen-binding fragment thereof, such as secukinumab) is administered to the patient IV at a dose of about 6 mg/kg once during week 0, and thereafter, as an IV dose of about 3 mg/kg every 4 weeks (monthly), beginning during week 4. In this manner, the patient is dosed IV with about 6 mg/kg of the IL-17 antagonist (e.g., secukinumab) during weeks 0, and thereafter, as an IV dose of about 3 mg/kg during week 4, 8, 12, 16, 20, etc.

In some embodiments, the IL-17 antagonist (e.g., IL-17 antibody or antigen-binding fragment thereof, such as secukinumab) is IV administered to the patient (e.g., a patient having LPP, MLP, CLP or combinations thereof) at a dose of about 4 mg/kg-about 9 mg/kg (preferably about 6 mg/kg) once during week 0, and thereafter, an IV dose of about 2.0-about 4 mg/kg (preferably about 3 mg/kg) every 8 weeks (every other month), beginning during week 4.

In some embodiments, it is contemplated that the IL-17 antagonist (e.g., IL-17 antibody or antigen-binding fragment thereof, such as secukinumab) may be IV administered to the patient (e.g., a patient having LPP, MLP, CLP or combinations thereof) at a dose of about 10 mg/kg monthly (every 4 weeks). In some embodiments, it is contemplated that the IL-17 antagonist (e.g., IL-17 antibody or antigen-binding fragment thereof, such as secukinumab) may be IV administered to the patient (e.g., a patient having LPP, MLP, CLP or combinations thereof) at a dose of about 10 mg/kg every two months (every 8 weeks). In some embodiments, it is contemplated that the IL-17 antagonist (e.g., IL-17 antibody or antigen-binding fragment thereof, such as secukinumab) may be IV administered to the patient (e.g., a patient having LPP, MLP, CLP or combinations thereof) at a dose of about 10 mg/kg monthly (every 4 weeks) during week 0, 4, 8, and thereafter at a dose of about 10 mg/kg (e.g., 10 mg/kg) every two months (every 8 weeks), beginning during week 16.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient (e.g., a patient having LPP, MLP, CLP or combinations thereof) without a loading regimen, e.g., the antagonist may be administered to the patient SC at about 150 mg-about 300 mg (e.g., about 150 mg, about 300 mg) every four weeks. In this manner, the patient is dosed SC with about 150 mg-about 300 mg (e.g., about 150 mg, about 300 mg) of the IL-17 antagonist (e.g., secukinumab) during weeks 0, 4, 8, 12, 16, 20, etc.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient (e.g., a patient having LPP, MLP, CLP or combinations thereof) without a loading regimen, e.g., the antagonist may be administered to the patient IV at about 2.5-about 4 mg/kg (preferably about 3 mg/kg) every month or at about 2.5-about 4 mg/kg (preferably about 3 mg/kg) every two months.

Alternatively, the IL-17 antagonists, e.g., IL-17 antibodies, e.g., secukinumab, can also be delivered orally (e.g., into the intestinal lumen using Rani Therapeutics technology, e.g., technology set forth in U.S. Pat. Nos. 8,734,429; 9,492,378; 9,456,988; 9,415,004; 9,6297,99; 9,757,548; 9,757,514; 9,402,806; US Pub. Appln. 2017/0189659, 2017/0100459).

It will be understood that dose escalation may be required for certain patients, e.g., a patient having LPP, MLP, CLP or combinations thereof who display inadequate response (e.g., partial response, failed response, or loss of response over time, e.g., as measured by any of the LP scoring systems disclosed herein) to treatment with the IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof) by week 10, week 12, week 14, week 16, week 18, week 20, week 22, week 24, week 48, week 52, or week 104 of treatment. Thus, SC dosages of secukinumab may be greater than about 150 mg-about 300 mg SC, e.g., about 200 mg, about 250 mg (in the case of an original 150 mg dose), about 350 mg, about 450 mg (in the case of an original 300 mg dose), etc.; similarly, IV dosages may be greater than about 2 mg/kg-about 9 mg/kg, e.g., about 2.5 mg/kg, about 3 mg/kg, 4 mg/kg, about 5 mg/kg, about 6 mg/kg (e.g., in the case of an original 2 mg/kg dose), about 9.5 mg/kg, 10 mg/kg, 11 mg/kg, 12 mg/kg, 15 mg/kg, 20 mg/kg, 25 mg/kg, 30 mg/kg, 35 mg/kg (in the case of an original 9 mg/kg mg dose), etc.

Similarly, more frequent dosing may be used (e.g., during the maintenance regimen) in certain patients, e.g., a patient having an inadequate response (e.g., partial response, failed response, or loss of response over time) to treatment with the IL-17 antibody or antigen-binding fragment thereof, e.g., secukinumab. These patients may be switched to more frequent administration (rather than increased dose), e.g., switched from administration of the IL-17 antibody or antigen-binding fragment thereof, e.g., secukinumab, every 4 weeks (monthly; Q4w) to administration every two weeks (Q2w) or every week (Q1w), or from administration every 2 weeks (Q2w) to administration every week (Q1w). This switch may be done as determined necessary by a health care professional, e.g., at week 10, week 12, week 14, week 16, week 18, week 20, week 22, week 24, week 48, week 52, or week 104 of treatment.

Thus, in some embodiments, if the patient does not adequately respond to treatment with the IL-17 antibody or antigen-binding fragment thereof (e.g., secukinumab) following a period of every four week administration, then the IL-17 antibody or antigen-binding fragment thereof (e.g., secukinumab) is administered to the patient every two weeks (Q2w) as a maintenance regimen. In the aforementioned embodiments, the “period of every four week administration” is determined by a health care professional based on patient response. For example, assuming an induction regimen (e.g., SC induction, e.g., using 150 mg or 300 mg secukinumab) dosing during week 0, 1, 2, 3, and 4, with a first Q4w maintenance dose at week 8, a health care professional may switch a patient from Q4w to Q2w maintenance treatment with the first Q2w administration occurring at week 10, week 14, week 18, week 22, week 26, week 30, week 54, etc. As another example, assuming an induction regimen (e.g., SC induction, e.g., using 150 mg or 300 mg secukinumab) of weekly dosing during week 0, 1, 2, 3, and 4, with a first Q4w maintenance dose at week 8, a health care professional may switch a patient from Q4w to Q2w maintenance treatment with the first Q2w administration occurring by week 12, week 16, week 20, week 24, week 28, week 52, etc.

It will also be understood that dose reduction may also be used for certain patients, e.g., a patient having LP, MLP, CLP or combinations thereof who displays a particularly robust treatment response, or an adverse event/response to treatment with the IL-17 antagonist (e.g., IL-17 antibody or antigen-binding fragment thereof, e.g., secukinumab). Thus, dosages of the IL-17 antagonist (e.g., IL-17 antibody or antigen-binding fragment thereof, e.g., secukinumab), may be lowered to less than about 150 mg-about 300 mg SC, e.g., about 250 mg, about 200 mg, about 150 mg (in the case of an original 300 mg dose); about 100 mg, about 50 mg (in the case of an original 150 mg dose), etc. Similarly, IV dosages may be lowered to less than about 8 mg/kg, e.g., about 7 mg/kg, 5 mg/kg, 4 mg/kg, 3 mg/kg, 2 mg/kg, 1 mg/kg, etc. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose of 300 mg or 150 mg delivered SC, and the dose is then escalated to about 450 mg (in the case of an original 300 mg dose) or about 300 mg (in the case of an original 150 mg dose) if needed, as determined by a health care professional.

Similarly, less frequent dosing may be used during the maintenance regimen in certain patients, e.g., a patient having a particularly robust treatment response, or an adverse event/response to treatment with the IL-17 antibody or antigen-binding fragment thereof, e.g., secukinumab. These patients may be switched to less frequent administration (rather than decreased dose), e.g., switched from administration of the IL-17 antibody or antigen-binding fragment thereof, e.g., secukinumab, every 4 weeks (monthly; Q4w) to administration every six weeks (Q6w) or eight weeks (Q8w), or from administration of the IL-17 antibody or antigen-binding fragment thereof, e.g., secukinumab, every 2 weeks (monthly; Q2w) to administration every four weeks (Q4w) or every six weeks (Q6w). This switch may be done as determined necessary by a health care professional, e.g., at week 10, week 12, week 14, week 16, week 18, week 20, week 22, week 24, week 48, week 52, or week 104 of treatment.

Thus, in some embodiments, if the patient adequately responds to treatment with the IL-17 antibody or antigen-binding fragment thereof (e.g., secukinumab) following a period of every two week (Q2w) administration, then the IL-17 antibody or antigen-binding fragment thereof (e.g., secukinumab) is administered to the patient every four weeks (Q4w) as a maintenance regimen. In the aforementioned embodiments, the “period of every four week administration” is determined by a health care professional based on patient response. For example, assuming an induction regimen (e.g., SC induction, e.g., using 150 mg or 300 mg secukinumab) dosing during week 0, 1, 2, 3, and 4, with a first Q2w maintenance dose at week 6, a health care professional may switch a patient from Q2w to Q4w maintenance treatment with the first Q4w administration occurring at week 8, week 10, week 12, week 14, week 16, week 18, week 20, week 22, week 24, week 52, etc. As another example, assuming an induction regimen (e.g., SC induction, e.g., using 150 mg or 300 mg secukinumab) of weekly dosing during week 0, 1, 2, 3, and 4, with a first Q2w maintenance dose at week 6, a health care professional may switch a patient from Q24w to Q4w maintenance treatment with the first Q4w administration occurring by week 8, week 10, week 12, week 14, week 16, week 18, week 20, week 22, week 24, week 52, etc.

As used herein, “fixed dose” refers to a flat dose, i.e., a dose that is unchanged regardless of a patient's characteristics. Thus, a fixed dose differs from a variable dose, such as a body-surface area-based dose or a weight-based dose (typically given as mg/kg). In some embodiments of the disclosed methods, uses, pharmaceutical compositions, kits, etc., the LP (e.g., CLP, MLP, LPP, or combinations thereof) patient is administered fixed doses of the IL-17 antibody, e.g., fixed doses of secukinumab, e.g., fixed doses of about 75 mg-about 450 mg secukinumab, e.g., about 75 mg, about 150 mg, about 300 mg, about 400 mg or about 450 mg secukinumab. Alternatively, in some embodiments, the LP (e.g., CLP, MLP, LPP, or combinations thereof) patient is administered a weight-based dose, e.g., a dose given in mg based on patient weight in kg (mg/kg).

The timing of dosing is generally measured from the day of the first dose of secukinumab (which is also known as “baseline”). However, health care providers often use different naming conventions to identify dosing schedules, as shown in Table 4.

TABLE 4
Common naming conventions for dosing regimens.
Bolded items refer to the naming convention used herein.

| Week | 0/1 | 1/2 | 2/3 | 3/4 | 4/5 | 5/6 | 6/7 | 7/8 | 8/9 | 9/10 | 10/11 | etc |
|---|---|---|---|---|---|---|---|---|---|---|---|---|
| 1^{st} | 0/1 | 7/8 | 14/15 | 21/22 | 28/29 | 35/36 | 42/43 | 49/50 | 56/57 | 63/64 | 70/71 | etc. |
| day | | | | | | | | | | | | |
| of | | | | | | | | | | | | |
| week | | | | | | | | | | | | |

Notably, week zero may be referred to as week one by some health care providers, while day zero may be referred to as day one by some health care providers. Thus, it is possible that different health care professionals will designate, e.g., a dose as being given during week 3/on day 21, during week 3/on day 22, during week 4/on day 21, during week 4/on day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the IL-17 antibody regardless of whether the health care professional refers to a particular week as “week 1” or “week 2”.

In a one dosing regimen, the antibody is administered during week 0, 1, 2, 3, 4, 8, 12, 16, 20, etc. Some providers may refer to this regimen as weekly for five weeks and then monthly (or every 4 weeks) thereafter, beginning during week 8, while others may refer to this regimen as weekly for four weeks and then monthly (or every 4 weeks) thereafter, beginning during week 4. It will be appreciated by a skilled artisan that administering a patient an injection at weeks 0, 1, 2 and 3, followed by once monthly dosing starting at week 4 is the same as: 1) administering the patient an injection at weeks 0, 1, 2, 3, and 4, followed by once monthly dosing starting at week 8; 2) administering the patient an injection at weeks 0, 1, 2, 3 and 4 followed by dosing every 4 weeks; and 3) administering the patient an injection at weeks 0, 1, 2, 3 and 4 followed by monthly administration.

In one embodiment, the antibody is administered to a LP patient during week 0, 1, 2, 3, 4, 6, 8, 10, 12, etc. Some providers may refer to this regimen as weekly for five weeks and then every other week (or every 2 weeks) thereafter, beginning during week 6, while others may refer to this regimen as weekly for four weeks and then every other week (or every 2 weeks) thereafter, beginning during week 4. It will be appreciated by a skilled artisan that administering a patient an injection at weeks 0, 1, 2 and 3, followed by administration every other week (or every 2 weeks) starting at week 4 is the same as: 1) administering the patient an injection at weeks 0, 1, 2, 3, and 4, followed by dosing every other week (or every 2 weeks) starting at week 6; 2) administering the patient an injection at weeks 0, 1, 2, 3 and 4 followed by dosing every 2 weeks; and 3) administering the patient an injection at weeks 0, 1, 2, 3 and 4 followed by every other week administration.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., SC or IV). As an example, if a desired SC dose is 300 mg, then a clinician may use 2 ml of an IL-17 antibody formulation having a concentration of 150 mg/ml, 1 ml of an IL-17 antibody formulation having a concentration of 300 mg/ml, 0.5 ml of an IL-17 antibody formulation having a concentration of 600 mg/ml, etc. In each such case, these IL-17 antibody formulations are at a concentration high enough to allow subcutaneous delivery of the IL-17 antibody. Subcutaneous delivery typically requires delivery of volumes of less than or equal to about 2 ml, preferably a volume of about 1 ml or less. Preferred formulations are ready-to-use liquid pharmaceutical compositions comprising about 25 mg/mL to about 150 mg/mL secukinumab, about 10 mM to about 30 mM histidine pH 5.8, about 200 mM to about 225 mM trehalose, about 0.02% polysorbate 80, and about 2.5 mM to about 20 mM methionine.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 300 mg, then a clinician may use 2 mL from a container that contains an IL-17 antibody formulation with a concentration of 150 mg/mL, 1 mL from a container that contains an IL-17 antibody formulation with a concentration of 300 mg/mL, 0.5 mL from a container contains an IL-17 antibody formulation with a concentration of 600 mg/ml, etc. In each such case, these containers have a sufficient amount of the IL-17 antagonist to allow delivery of the desired 300 mg dose.

In some embodiments of the disclosed uses, methods, and kits, the dose of the IL-17 antibody (e.g., secukinumab) or an antigen-binding fragment thereof is about 300 mg, the IL-17 antibody (e.g., secukinumab) or an antigen-binding fragment thereof is comprised in a liquid pharmaceutical formulation at a concentration of 150 mg/ml, and 2 ml of the pharmaceutical formulation is disposed within two pre-filled syringes, injection pens, or autoinjectors, each having 1 ml of the pharmaceutical formulation. In this case, the patient receives two injections of 1 ml each, for a total dose of 300 mg, during each administration. In some embodiments, the dose of the IL-17 antibody (e.g., secukinumab) or an antigen-binding fragment thereof is about 300 mg, the IL-17 antibody (e.g., secukinumab) or an antigen-binding fragment thereof is comprised in a liquid pharmaceutical formulation at a concentration of 150 mg/ml, and 2 ml of the pharmaceutical formulation is disposed within an autoinjector or PFS. In this case, the patient receives one injection of 2 ml, for a total dose of 300 mg, during each administration. In methods employing one injection of 2 ml (e.g., via a single PFS or autoinjector) (i.e., a “single-dose preparation”), the drug exposure (AUC) and maximal concentration (Cₘₐₓ) is equivalent (similar to, i.e., within the range of acceptable variation according to US FDA standards) to methods employing two injections of 1 ml (e.g., via two PFSs or two AIs) (i.e., a “multiple-dose preparation”).

Disclosed herein are methods of treating lichen planopilaris (LPP), comprising subcutaneously (SC) administering to a patient in need thereof a dose of about 150 mg-about 300 mg of an Interleukin (IL)-17 antibody, or an antigen-binding fragment thereof, weekly during weeks 0, 1, 2, 3, and 4, and every four weeks thereafter, beginning during week 8, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

Also disclosed herein is an IL-17 antibody (e.g. secukinumab) or an antigen-binding fragment thereof, for use in treating LPP, which is to be subcutaneously (SC) administered to a patient in need thereof at a dose of about 150 mg-about 300 mg weekly during weeks 0, 1, 2, 3, and 4, and thereafter SC at a dose of about 150 mg-about 300 mg monthly (every 4 weeks), beginning during week 8. Also disclosed herein is an IL-17 antibody (e.g. secukinumab) or an antigen-binding fragment thereof, for use in the manufacture of a medicament for treating LPP, which is to be subcutaneously (SC) administered to a patient in need thereof at a dose of about 150 mg-about 300 mg weekly during weeks 0, 1, 2, 3, and 4, and thereafter SC at a dose of about 150 mg-about 300 mg monthly (every 4 weeks), beginning during week 8.

Disclosed herein are methods of treating of treating lichen planus (LP), comprising subcutaneously (SC) administering to a patient in need thereof a dose of about 150 mg-about 300 mg of an Interleukin (IL)-17 antibody, or an antigen-binding fragment thereof, weekly during weeks 0, 1, 2, 3, and 4, and every two weeks thereafter, beginning during week 6, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

Also disclosed herein is an IL-17 antibody (e.g. secukinumab) or an antigen-binding fragment thereof, for use in treating LP, which is to be subcutaneously (SC) administered to a patient in need thereof at a dose of about 150 mg-about 300 mg weekly during weeks 0, 1, 2, 3, and 4, and thereafter SC at a dose of about 150 mg-about 300 mg every 2 weeks, beginning during week 6. Also disclosed herein is an IL-17 antibody (e.g. secukinumab) or an antigen-binding fragment thereof, for use in the manufacture of a medicament for treating LPP, which is to be subcutaneously (SC) administered to a patient in need thereof at a dose of about 150 mg-about 300 mg weekly during weeks 0, 1, 2, 3, and 4, and thereafter SC at a dose of about 150 mg-about 300 mg every 2 weeks, beginning during week 6.

Disclosed herein are methods of treating of treating lichen planus (LP), comprising intravenously (IV) administering to a patient in need thereof a dose of about 4 mg/kg-about 9 mg/kg (preferably about 6 mg/kg) of an Interleukin (IL)-17 antibody, or an antigen-binding fragment thereof, once during week 0, and thereafter administering an IV dose of about 2 mg/kg-about 4 mg/kg (preferably about 3 mg/kg) of the IL-17 antibody, or an antigen-binding fragment thereof every four weeks, beginning during week 4, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

Also disclosed herein is an IL-17 antibody (e.g. secukinumab) or an antigen-binding fragment thereof, for use in treating LP, which is to be intravenously (IV) administered to a patient in need thereof at a dose of about 4 mg/kg to about 9 mg/kg (preferably about 6 mg/kg) once during week 0, and thereafter IV at a dose of about 2 mg/kg to about 4 mg/kg (preferably about 3 mg/kg) monthly (every 4 weeks), beginning during week 4. Also disclosed herein is an IL-17 antibody (e.g. secukinumab) or an antigen-binding fragment thereof, for use in the manufacture of a medicament for treating LPP, which is to be intravenously (IV) administered to a patient in need thereof at a dose of about 4 mg/kg to about 9 mg/kg (preferably about 6 mg/kg) once during week 0, and thereafter IV at a dose of about 2 mg/kg to about 4 mg/kg (preferably about 3 mg/kg) monthly (every 4 weeks), beginning during week 4.

In some embodiments of the disclosed methods, uses, compositions and kits, the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE), and wherein the IL-17 antibody has an in vivo half-life of about 23 to about 30 days.

In some embodiments of the disclosed methods, uses, compositions and kits, if the patient does not adequately respond to treatment with the IL-17 antibody or antigen-binding fragment thereof following a period of every four week administration, then the IL-17 antibody or antigen-binding fragment thereof is administered to the patient every two weeks as a maintenance regimen.

In some embodiments of the disclosed methods, uses, compositions and kits, the dose of IL-17 antibody or antigen-binding fragment thereof is 150 mg.

In some embodiments of the disclosed methods, uses, compositions and kits, the dose of IL-17 antibody or antigen-binding fragment thereof is 300 mg.

In some embodiments of the disclosed methods, uses, compositions and kits, prior to treatment with the IL-17 antibody or antigen-binding fragment thereof, the patient did not adequately respond to treatment with a lichenoid therapy selected from the group consisting of a topical therapy, a systemic therapy, phototherapy, a retinoid, and any combination thereof.

In some embodiments of the disclosed methods, uses, compositions and kits, prior to treatment with the IL-17 antibody or antigen-binding fragment thereof, the patient was refractory to topical corticosteroid therapy or the patient did not adequately respond to treatment with a topical steroid.

In some embodiments of the disclosed methods, uses, compositions and kits, during treatment with the IL-17 antibody or antigen-binding fragment thereof, the patient is concomitantly administered a lichenoid therapy selected from the group consisting of a topical therapy, a systemic therapy, phototherapy, a retinoid, and any combination thereof.

In some embodiments of the disclosed methods, uses, compositions and kits, during treatment with the IL-17 antibody or antigen-binding fragment thereof, the patient is concomitantly administered at least one low to medium potency topical steroid.

In some embodiments of the disclosed methods, uses, compositions and kits, the patient has biopsy-confirmed cutaneous lichen planus (CLP), biopsy-confirmed mucosal lichen planus (MLP) or biopsy-confirmed lichen planopilaris (LPP).

In some embodiments of the disclosed methods, uses, compositions and kits, the patient has CLP and a baseline Body Surface Area (BSA) involvement of ≥3%, with or without nail involvement.

In some embodiments of the disclosed methods, uses, compositions and kits, the patient has MLP and one or more affected locations selected from the oral cavity, genitals, and conjunctiva.

In some embodiments of the disclosed methods, uses, compositions and kits, the patient has LPP and at least three active patches.

In some embodiments of the disclosed methods, uses, compositions and kits, the patient:

a) has a baseline Investigator's Global Assessment (IGA) of ≥3; and

b) is refractory to topical corticosteroid therapy or has had an inadequate response to topical steroids.

In some embodiments of the disclosed methods, uses, compositions and kits, following treatment with the IL-17 antibody or antigen-binding fragment thereof, the patient achieves at least two points improvement in IGA score versus baseline IGA score.

In some embodiments of the disclosed methods, uses, compositions and kits, the patient is an adult.

In some embodiments of the disclosed methods, uses, compositions and kits, the IL-17 antibody or antigen-binding fragment thereof is disposed in a pharmaceutical formulation, wherein said pharmaceutical formulation further comprises a buffer and a stabilizer.

In some embodiments of the disclosed methods, uses, compositions and kits, the pharmaceutical formulation is in liquid form.

In some embodiments of the disclosed methods, uses, compositions and kits, the pharmaceutical formulation is in lyophilized form.

In some embodiments of the disclosed methods, uses, compositions and kits, the pharmaceutical formulation is disposed within at least one pre-filled syringe, at least one vial, at least one injection pen, or at least one autoinjector.

In some embodiments of the disclosed methods, uses, compositions and kits, the at least one pre-filled syringe, at least one vial, at least one injection pen, or at least one autoinjector is disposed within a kit, and wherein said kit further comprises instructions for use.

In some embodiments of the disclosed methods, uses, compositions and kits, the dose of the IL-17 antibody or antigen-binding fragment is 300 mg, which is administered to the patient as a single subcutaneous administration in a total volume of 2 milliliters (mL) from a formulation comprising 150 mg/ml of the IL-17 antibody or antigen-binding fragment, and wherein the pharmacological exposure of the patient to the IL-17 antibody or antigen-binding fragment is equivalent to the pharmacological exposure of the patient to the IL-17 antibody or antigen-binding fragment using two separate subcutaneous administrations of a total volume of 1 ml each of the same formulation.

In some embodiments of the disclosed methods, uses, compositions and kits, the dose of the IL-17 antibody or antigen-binding fragment administered to the patient is 300 mg, which is administered as two separate subcutaneous administrations in a volume of 1 mL each from a formulation comprising 150 mg/ml of the IL-17 antibody or antigen-binding fragment

In some embodiments of the disclosed methods, uses, compositions and kits, the IL-17 antibody or antigen-binding fragment has a Tₘₐₓ of about 7-8 days.

In some embodiments of the disclosed methods, uses, compositions and kits, the IL-17 antibody or antigen-binding fragment has an absolute bioavailability of about 60%-about 80%.

In some embodiments of the disclosed methods, uses, compositions and kits, the IL-17 antibody or antigen-binding fragment is a human monoclonal antibody.

In some embodiments of the disclosed methods, uses, compositions and kits, the IL-17 antibody or antigen-binding fragment is of the IgG₁/kappa isotype.

In some embodiments, when the method is used to treat a population of patients, at least 30% of said patients achieve an IGA 0/1 after 16 weeks of treatment.

In some embodiments, when the method is used to treat a population of patients, at least 40% of said patients achieve an IGA 0/1 after 16 weeks of treatment.

In some embodiments of the disclosed methods, uses, compositions and kits, the patient has MLP, and the patient achieves an improvement in MLP as measured by the Modified Oral Mucositis Index (MOMI) following 16 weeks of treatment with the IL-17 antibody or antigen-binding fragment thereof.

In some embodiments of the disclosed methods, uses, compositions and kits, the patient achieves an improvement in LPP as measured by the Lichen Planopilaris Activity Index (LPPAI) following 16 weeks of treatment with the IL-17 antibody or antigen-binding fragment thereof.

In some embodiments of the disclosed methods, uses, compositions and kits, the patient achieves an improvement in pruritus as measured by the Peak Pruritus Numerical Rating Scale (NRS) following 16 weeks of treatment with the IL-17 antibody or antigen-binding fragment thereof.

In some embodiments of the disclosed methods, uses, compositions and kits, the patient achieves an improvement in pain as measured by a Visual Analogue Scale (VAS) following 16 weeks of treatment with the IL-17 antibody or antigen-binding fragment thereof.

In some embodiments of the disclosed methods, uses, compositions and kits, the patient achieves an improvement in quality of life as measured by the Dermatology Life Quality Index (DLQI) 0/1 following 16 weeks of treatment with the IL-17 antibody or antigen-binding fragment thereof.

In some embodiments of the disclosed methods, uses, compositions and kits, the patient has MLP, and the patient achieves an improvement in MLP as measured the Oral Health Impact Profile (OHIP-14) following 16 weeks of treatment with the IL-17 antibody or antigen-binding fragment thereof.

In some embodiments of the disclosed methods, uses, compositions and kits, the patient is treated with the IL-17 antibody or antigen-binding fragment thereof for at least one year.

In preferred embodiments of the disclosure, the IL-17 antibody or antigen-binding fragment thereof is a monoclonal antibody.

In preferred embodiments of the disclosure, the IL-17 antibody or antigen-binding fragment thereof is a human or humanized antibody.

In preferred embodiments of the disclosure, the IL-17 antibody or antigen-binding fragment thereof is a human antibody.

In preferred embodiments of the disclosed methods, uses and kits, the IL-17 antibody or antigen-binding fragment is a human monoclonal antibody.

In preferred embodiments of the disclosure, the IL-17 antibody or antigen-binding fragment thereof is a human antibody of the IgG₁ subtype.

In preferred embodiments the IL-17 antibody or antigen-binding fragment thereof has a kappa light chain.

In preferred embodiments of the disclosure, the IL-17 antibody or antigen-binding fragment thereof is a human antibody of the IgG₁ kappa type.

In preferred embodiments of the disclosed methods, uses and kits, the IL-17 antibody or antigen-binding fragment has a Tₘₐₓ of about 7-8 days.

In preferred embodiments of the disclosed methods, uses and kits, the IL-17 antibody or antigen-binding fragment has an absolute bioavailability of about 60%-about 80%.

In preferred embodiments of the disclosure, the IL-17 antibody or antigen-binding fragment thereof is secukinumab.

Disclosed herein are methods of treating of treating an adult patient with lichen planopilaris (LPP) that is inadequately controlled with topical corticosteroid therapy or for whom topical corticosteroid therapy is not advisable, comprising administering a dose of about 300 mg secukinumab subcutaneously to said patient during week 0, 1, 2, 3, and 4, and then every four weeks thereafter. In preferred embodiments, the LPP is biopsy-confirmed.

Disclosed herein are methods of treating of treating an adult patient with lichen planus (LP) inadequately controlled with topical corticosteroid therapy or for whom topical corticosteroid therapy is not advisable, comprising administering a dose of about 300 mg secukinumab subcutaneously to said patient during week 0, 1, 2, 3, and 4, and then every two weeks thereafter. In preferred embodiments, the patient has cutaneous lichen planus (CLP), mucosal lichen planus (MLP), lichen planopilaris (LPP), or any combination thereof. In some embodiments of the disclosed methods, uses, compositions and kits, the CLP, MLP or LPP is biopsy-confirmed.

Disclosed herein are methods of treating of treating an adult patient with lichen planus (LP) inadequately controlled with topical corticosteroid therapy or for whom topical corticosteroid therapy is not advisable, comprising, intravenously (IV) administering to the patient a dose of about 6 mg/kg secukinumab once during week 0, and thereafter administering an IV dose of about 3 mg/kg secukinumab every four weeks, beginning during week 4. In preferred embodiments, the patient has cutaneous lichen planus (CLP), mucosal lichen planus (MLP), lichen planopilaris (LPP), or any combination thereof. In some embodiments of the disclosed methods, uses, compositions and kits, the CLP, MLP or LPP is biopsy-confirmed.

### Kits

The disclosure also encompasses kits for treating LP. Such kits comprise an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) (e.g., in liquid or lyophilized form) or a pharmaceutical composition comprising the IL-17 antagonist (described supra). Additionally, such kits may comprise means for administering the IL-17 antagonist (e.g., an autoinjector, a syringe and vial, a prefilled syringe, a prefilled pen) and instructions for use. These kits may contain additional therapeutic HS agents (described supra) for treating LP (e.g., CLP, LPP, MLP, or combinations thereof), e.g., for delivery in combination with the enclosed IL-17 antagonist, e.g., IL-17 binding molecule, e.g., IL-17 antibody, e.g., secukinumab. Such kits may also comprise instructions for administration of the IL-17 antagonist (e.g., IL-17 antibody, e.g., secukinumab) to treat the patient having LP (e.g., CLP, LPP, MLP, or combinations thereof). Such instructions may provide the dose (e.g., 3 mg/kg, 6 mg/kg, 300 mg, 450 mg), route of administration (e.g., IV, SC), and dosing regimen (e.g., weekly, monthly, weekly and then monthly, weekly and then every other week, etc.) for use with the enclosed IL-17 antagonist, e.g., IL-17 binding molecule, e.g., IL-17 antibody, e.g., secukinumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a health care professional) or a medical practitioner may administer the drug. In some embodiments, a total dose of 300 mg is to be delivered in a total volume of 2 ml, which is disposed in two PFSs or autoinjectors, each PFS or autoinjector containing a volume of 1 ml having 150 mg/ml of the IL-17 antibody, e.g., secukinumab. In this case, the patient receives two 1 ml injections (a multi-dose preparation). In preferred embodiments, a total dose of 300 mg is to be delivered in a total volume of 2 ml having 150 mg/ml of the IL-17 antibody, e.g., secukinumab, which is disposed in a single PFS or autoinjector. In this case, the patient receives one 2 ml injection (a single dose preparation).

Disclosed herein are kits for use treating a patient having LP (e.g., CLP, LPP, MLP, or combinations thereof), comprising an IL-17 antagonist (e.g., IL-17 binding molecule, e.g., IL-17 antibody or antigen-binding fragment thereof, e.g., secukinumab) and means for administering the IL-17 antagonist to the patent having LP (e.g., CLP, LPP, MLP, or combinations thereof).

In some embodiments, the kit further comprises instructions for administration of the IL-17 antagonist to a patient (preferably a patient having LPP), wherein the instructions indicate that the IL-17 antagonist (e.g., IL-17 binding molecule, e.g., IL-17 antibody or antigen-binding fragment thereof, e.g., secukinumab) is to be administered to the patient SC at a dose of about 150 mg-about 300 mg (e.g., about 150 mg, about 300 mg) weekly during week 0, 1, 2, 3, and 4 and then every four weeks thereafter.

In some embodiments, the kit further comprises instructions for administration of the IL-17 antagonist to a patient having LP (e.g., LPP, CLP, MLP, or combinations thereof), wherein the instructions indicate that the IL-17 antagonist (e.g., IL-17 binding molecule, e.g., IL-17 antibody or antigen-binding fragment thereof, e.g., secukinumab) is to be administered to the patient SC at a dose of about 150 mg-about 300 mg (e.g., about 150 mg, about 300 mg) weekly during week 0, 1, 2, 3, and 4 and then every two weeks thereafter.

In some embodiments, the kit further comprises instructions for administration of the IL-17 antagonist to a patient having LP, wherein the instructions indicate that the IL-17 antagonist (e.g., IL-17 binding molecule, e.g., IL-17 antibody or antigen-binding fragment thereof, e.g., secukinumab) is to be IV administered to the patient at a dose of about 4 mg/kg-about 9 mg/kg (preferably about 6 mg/kg) once during week 0, and thereafter, as an IV dose of about 2-about 4 mg/kg (preferably about 3 mg/kg) every 4 weeks (monthly), beginning during week 4.

### General

In preferred embodiments of the disclosed uses, methods and kits, the IL-17 antibody or antigen-binding fragment thereof is selected from the group consisting of: a) an IL-17 antibody or antigen-binding fragment thereof that binds to an epitope of human IL-17 comprising Leu74, Tyr85, His86, Met87, Asn88, Val124, Thr125, Pro126, Ile127, Val128, His129; b) an IL-17 antibody or antigen-binding fragment thereof that binds to an epitope of human IL-17 comprising Tyr43, Tyr44, Arg46, Ala79, Asp80; c) an IL-17 antibody or antigen-binding fragment thereof that binds to an epitope of an IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 23 to about 35 days; e) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., Biacore®), and wherein the IL-17 antibody has an in vivo half-life of about 23 to about 30 days; and f) an IL-17 antibody or antigen-binding fragment thereof comprising: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO:15; or xi) an immunoglobulin light chain comprising the amino acid sequence set forth as SEQ ID NO:14 and an immunoglobulin heavy chain comprising the amino acid sequence set forth as SEQ ID NO:15.

In the most preferred embodiments of the disclosed methods, kits, or uses, the IL-17 antibody or antigen-binding fragment thereof is a monoclonal antibody, preferably a human antibody, preferably a human IgG₁ antibody, most preferably secukinumab.

In the most preferred embodiments of the disclosed methods, kits, or uses, the dose size of the IL-17 antibody or antigen-binding fragment thereof (preferably secukinumab) is flat, the dose is 150 mg or 300 mg (most preferably 300 mg), the route of administration is SC, and the regimen is administration at week 0, 1, 2, 3, 4, 8, 12 etc. (weekly during week 0, 1, 2, 3, and 4, and then every four weeks, beginning during week 8) or administration at week 0, 1, 2, 3, 4, 6, 8, 10, 12 etc. (weekly during week 0, 1, 2, 3, and 4, and then every other week, beginning during week 6).

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

### EXAMPLES

### Example 1: Clinical Trial CAIN457S12201—a Proof of Concept Study to Evaluate the Efficacy, Safety and Tolerability of Secukinumab 300 mg Over 32 Week in Adult Patients with Biopsy-Confirmed Forms of Lichen Planus not Adequately Controlled with Topical Therapies

### Purpose and Rationale for Design:

The purpose of this proof of concept study is to study the efficacy of secukinumab in the treatment of adult patients with biopsy-certified lichen planus that is not adequately controlled with topical therapies, and to assess the safety and tolerability over 32 weeks of treatment. The double-blind, randomized, placebo-controlled design of this trial enables the evaluation of the efficacy and safety of secukinumab 300 mg in two different dosing regimens, and in three selected subtypes of lichen planus (MLP, CLP, LPP) in an adequate and controlled setting.

The rationale to assign the patients to a specific lichen planus subtype and to monitor them in a parallel-group fashion is to assess the efficacy and safety of secukinumab in each subtype individually. There is broad pathophysiological and clinical overlap between the three selected subtypes, especially between CLP and MLP subtypes, with many patients presenting overlapping symptoms and lesions. However, each subtype presents at different anatomical regions and with distinct clinical features, e.g., ulceration can be present in the mucosal subtype, but not in the cutaneous subtype, and hair follicle inflammation is the unique feature of lichen planopilaris.

Patients are divided into three subgroups, according to their predominant clinical subtype (confirmed by biopsy) in order to apply subtype-specific assessments. By using this design the trial is also able to collect data on the non-predominant (“concomitant”) subtype, e.g., data on cutaneous lesions in a patient who is enrolled in the predominantly mucosal LP subgroup. This “basket trial” design takes advantages of the overlap between the subtypes, by allowing enrollment and assessment of all three subtypes in one trial, while capturing the unique features of each subtype by using subtype-specific assessments, scores and endpoints.

### Study Design:

This is a multicenter, randomized, double-blind, placebo-controlled, parallel-group trial assessing the efficacy and safety of secukinumab 300 mg in two different dosing regimens in patients with biopsy-proven forms of lichen planus.

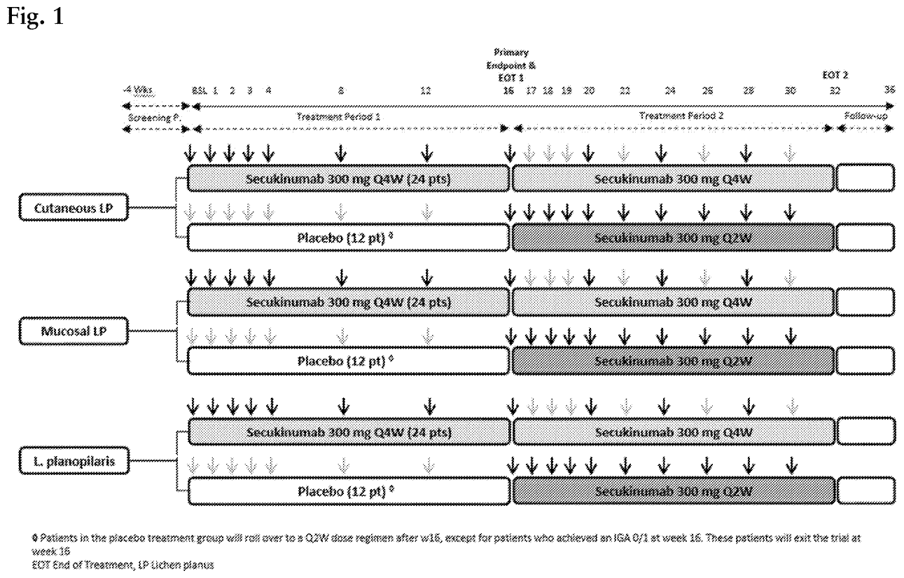

The study consists of three cohorts (one cohort per lichen planus subtype: cutaneous lichen planus (CLP), mucosal lichen planus (MLP) and lichen planopilaris (LPP)) and 4 study periods as illustrated in FIG. 1.

Patients are assigned to one of the three cohorts based on their predominant subtype and undergo a biopsy to confirm the clinical diagnosis at the screening visit:
Predominantly cutaneous lichen planus
Predominantly mucosal lichen planus
Lichen planopilaris

Each cohort will follow the same study design across the 4 periods:
Screening Period: up to 4 weeks prior to baseline
Treatment Period 1: baseline to Week 16
Treatment Period 2: Week 16 to Week 32
Follow-up: 8 weeks after Week 32

### Screening Period:

A screening period of up to 4 weeks is used to assess patient's eligibility for the trial and to washout/adjust prohibited medications. The screening period covers the time from the signature of informed consent/screening visit (−4 weeks) to the randomization visit (Week 0).

Patients can be re-screened if the patient fails the initial screening due to a transient condition or due to an insufficient prohibited medication washout period. Subjects can be re-screened only once and no re-screening procedure should be performed prior to re-consenting the subject.

### Treatment Period 1:

Treatment Period 1 is placebo-controlled and covers the time from Week 0 (randomization visit) to Week 16. Patients who meet all eligibility criteria are randomized in a 2:1 ratio to one of the following two treatment arms within their cohort:
Secukinumab 300 mg every 4 weeks arm: subjects receive a weekly induction treatment followed by secukinumab 300 mg every 4 weeks.
Placebo for 16 weeks followed by secukinumab 300 mg every 2 weeks arm: subjects receive matching placebo injections.

In Treatment Period 1 all subjects receive weekly subcutaneous injections of blinded study drug (either 300 mg secukinumab or placebo) at weeks 0, 1, 2, 3 and 4. Thereafter the frequency of blinded study drug injections for all subjects is every 4 weeks up to Week 16. Home administration of study drug is not allowed during Treatment Period 1. Subjects who complete Treatment Period 1 roll over to Treatment Period 2 at the week 16 visit. The only exception are subjects from the placebo for 16 weeks followed by secukinumab 300 mg every 2 weeks arm, who achieve spontaneous remission at the Week 16 visit. Spontaneous remission is defined as an IGA of 0 or 1 at Week 16. These subjects do not proceed to Treatment Period 2 to avoid unnecessary treatment. Instead they directly enter the Follow-up Period after the Week 16 visit.

### Treatment Period 2:

Treatment Period 2 starts at the Week 16 visit and covers the time until the Week 32 visit. Depending on the treatment arm, subjects receive the following treatments:
Secukinumab 300 mg every 4 weeks arm: subjects receive continued treatment with secukinumab 300 mg every 4 weeks plus matching placebo injections to maintain treatment blinding.
Placebo for 16 weeks followed by secukinumab 300 mg every 2 weeks arm: subjects are switched to active treatment with secukinumab 300 mg every 2 weeks including an induction starting at Week 16, with the exception of subjects achieving remission by Week 16.

The Week 16 injection is the first injection of Treatment Period 2.

Treatment remains blinded during Treatment Period 2. This means that starting at the Week 16 visit al subjects receive an induction consisting of weekly blinded study drug injections (either secukinumab 300 mg or placebo) at weeks 16, 17, 18, 19 and 20, followed by blinded study drug injections every 2 weeks, either secukinumab 300 mg alternating with placebo every 2 weeks (secukinumab 300 mg every 4 weeks arm) or secukinumab 300 mg every 2 weeks (placebo for 16 weeks followed by secukinumab 300 mg every 2 weeks arm) until week 30.

The last study drug injection is at Week 30. The end of Treatment Period 2 is Week 32. After Week 32, all subjects enter the Follow-up Period.

### Follow-Up:

There is an 8-week Follow-up Period after Week 32.

### Rationale for Dose and Regimen

Two different secukinumab dosing regimens will be evaluated in this study:
Secukinumab 300 mg s.c. every 4 weeks
Secukinumab 300 mg s.c. every 2 weeks

Both dosing regimens start with a regular induction consisting of weekly injections of secukinumab 300 mg at either weeks 0, 1, 2, 3, 4 or 16, 17, 18, 19, 20. As demonstrated in the extensive Phase 3 clinical development program for moderate-to-severe psoriasis, induction with weekly dosing during the first month is safe and enables fast achievement of effective drug concentrations, leading to a rapid onset of clinical response (Langley et al. (2014) N Engl J Med. 371(4):326-38).

### Rationale for 300 mg SC Every 4 Weeks Regimen:

Secukinumab 300 mg administered subcutaneously with an initial induction followed by administration every four weeks (maintenance) is in line with the secukinumab Phase 3 registration program, which has proven the efficacy and safety of this dosing regimen in patients with moderate to severe plaque-Psoriasis (Langley, 2014). Further, clinical evidence on the potential efficacy of secukinumab in patients with CLP and MLP derives from a case series showing clinical response to treatment with the 4-weekly dosing regimen (Solimani, 2019). No relevant safety issues were reported in this case series.

### Rationale for 300 mg SC Every 2 Weeks Regimen:

In addition to the 4 weekly dosing regimen outlined above, a second, higher dosing regimen, 300 mg every 2 weeks, is evaluated regarding efficacy and safety in patients with lichen planus in this Phase 2 study in order to compare two dosing regimens and assess the dose-response relationship.

Higher local exposure than in plaque-type psoriasis might be needed for successful treatment of moderate-to-severe lichen planus; this patient population can be highly resistant to topical treatments, especially in the case of ulcerative/erosive lesions. Recalcitrant patients then require systemic treatments, including immunosuppressive agents such as mycophenolate or cyclosporine, to achieve partial/full response (Deen (2015) J Dermatol. 42(3):311-4). There are only very few reports on the use of other biologic agents in lichen planus, one reporting the successful use of 40 mg adalimumab every week in a CLP patient (Chao (2009) Cutis 84(6):325-8).

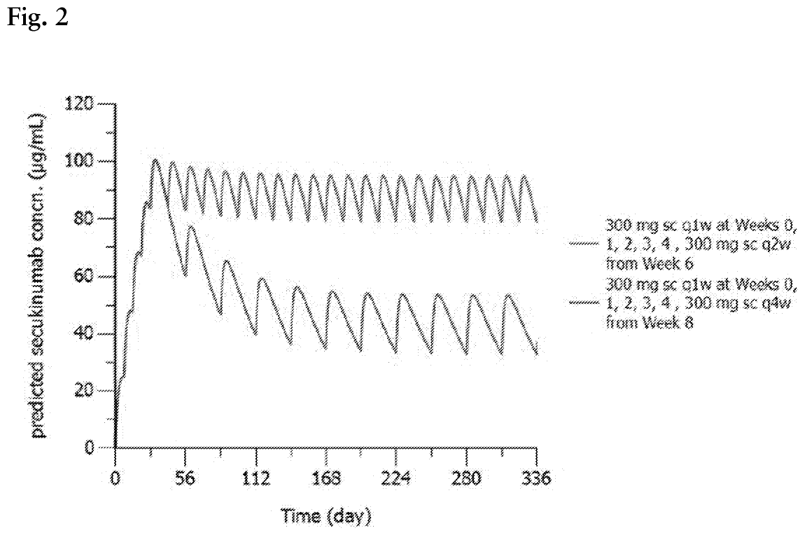

For secukinumab, after the same induction period (weekly during week 0, 1, 2, 3, and 4) during the first month, considerably higher and more consistent systemic exposure can be achieved with a shortened dose interval (every 2 weeks), than can be reached with the 4 weeks interval (FIG. 2). Secukinumab 300 mg s.c. every 2 weeks has been tested in approximately 120 patients for at least 24 weeks in completed clinical studies in uveitis and psoriasis. A safety profile in line with that of secukinumab 300 mg s.c. every 4 weeks has been observed. Furthermore, the 300 mg every 2 weeks dosing regimen is currently being evaluated in 2 Phase 3 trials in patients with Hidradenitis suppurativa (NCT03713632, NCT03713619) and in a Phase 3 trial in patients with moderate to severe psoriasis having a body weight ≥90 kg (NCT03504852).

A detailed protocol summary is below in Table 5:

| | |
|---|---|
| Full Title | A proof of concept study to evaluate the efficacy, safety and tolerability |
| | of secukinumab 300 mg over 32 weeks in adult patients with biopsy- |
| | proven forms of lichen planus not adequately controlled with topical |
| | therapies—PRELUDE |
| Brief title | Study of efficacy and safety of secukinumab 300 mg in adult patients |
| | with biopsy-proven forms of lichen planus |
| Purpose and | Lichen planus (LP) is an inflammatory disease affecting different |
| rationale | locations of the body (skin, oral cavity, genitalia, scalp, and nails). |
| | Current therapies are mostly symptomatic. However, 30-50% of |
| | patients are refractory to available therapies and experience high burden |
| | of disease due to lack of clinical control, as well as significant |
| | psychological discomfort and social disability resulting in profoundly |
| | impaired quality of life. |
| | This is a multicenter, randomized, double-blind, placebo-controlled, |
| | parallel-group trial assessing the efficacy and safety of secukinumab |
| | 300 mg in two different dosing regimens in patients with biopsy-proven |
| | forms of lichen planus, for which no systemic therapy is currently |
| | approved and which could be eligible for treatment with secukinumab. |
| Primary | The primary objective of this study is to evaluate the efficacy |
| Objective(s) | secukinumab when compared to placebo after 16 weeks of treatment, by |
| | comparing the proportion of patients achieveing Investigator's Global |
| | Assessment (IGA) response where IGA response is defined as |
| | achivement of absolute IGA score less or equal 2. |
| Secondary | Objective 1: To evaluate the efficacy of secukinumab 300 mg Q4W |
| Objectives | compared to placebo throughout 16 weeks in Treatment Period 1 and to |
| | evaluate the long term efficacy of secukinumab 300 mg Q4W |
| | throughout 32 weeks in Treatment Period 2 and evaluate the efficacy of |
| | secukinumab 300 mg Q2W in Treatment Period 2 by Investigator's |
| | Global Assessment (IGA), Dermatology Life Quality Index (DLQI), |
| | Physician Assessment of Surface Area of Disease (PS AD) for Skin |
| | Disease, Patient assessment of itch (NRS), Reticular Erythematous |
| | Ulcerative (REU) score, Oral Lichen Planus Symptoms Severity |
| | Measure (OLPSSM) score, Patient assessment of pain (NRS), LPP |
| | Activity Index (LPPAI), SCALPDEX Questionnaire. |
| | Objective 2: To assess the safety and tolerability of secukinumab in |
| | subjects with lichen planus by adverse events, laboratory values, vital |
| | signs from baseline to end of study visit. |
| Study design | This is a multicenter, randomized, double-blind, placebo-controlled, |
| | parallel-group study in approximately 108 patients with biopsy-proven |
| | lichen planus. The study consists of: Screening (up to 4 weeks), |
| | Treatment Period 1 (16 weeks), Treatment Period 2 (16 weeks). |
| | Subjects who prematurely discontinue the study, or who complete the |
| | study, will enter a post-treatment Follow-Up period (8 weeks). |
| Population | The study population will consist of adult subjects (18 years) with |
| | biopsy-proven lichen planus. It is planned to randomize approximately |
| | 108 subjects in approximately 36 study sites worldwide. At |
| | randomization, the subjects will be assigned to one of the 3 cohorts |
| | (predominant cutaneous lichen planus, predominant mucosal lichen |
| | planus and lichen planopilaris) according to the clinical features present. |
| Key Inclusion | 1. Written informed consent must be obtained before any assessment is |
| criteria | performed. |
| | 2. Female and male patients ≥18 years of age. |
| | 3. Subjects must have biopsy-confirmed forms of cutaneous lichen |
| | planus (CLP), mucosal lichen planus (MLP), or active lichen |
| | planopilaris (LPP) eligible for systemic therapy based on the following |
| | criteria: |
| | Rated IGA of ≥ 3 (moderate or severe) AND |
| | Inadequate response to topical corticosteroids of high-ultrahigh |
| | potency in the opinion of the investigator |
| Key Exclusion | 1. Clinical history suspicious for lichenoid drug eruption. |
| criteria | 2. Lichen planus pigmentosus. |
| | 3. Clinical picture or history suspicious of paraneoplastic mucosal |
| | lichen planus. |
| | 4. Subjects whose lichen planus is a predominantly bullous variant. |
| | 5. Mucosal lichen planus of the oral cavity or gastrointestinal |
| | involvement requiring the patient to use parenteral nutrition or feeding |
| | tube. |
| | 6. Clinical picture of burnt-out cicatricial alopecia (alopecia of Brocque) |
| | 7. Patients diagnosed with frontal fibrosing alopecia (FFA) without |
| | active patches of LPP. |
| | 8. Previous exposure to any other biologic drug directly targeting IL- |
| | 17A or IL-17RA (e.g. ixekizumab or brodalumab) or IL-23/p19 (e.g. |
| | tildrakizumab, guselkumab, risankizumab). |
| | 9. Hepatitis C antibody positive at screening unless viral load is 0. |
| Study treatment | 1. Secukinumab 300 mg, provided as 2 s.c. injections of 1 ml prefilled |
| | syringe (PFS). Each 1 ml syringe contains 150 mg secukinumab. |
| | 2. Placebo, provided as 2 s.c. injections of 1 ml prefilled syringe |
| | (PFS). |
| Efficacy | Efficacy assessments related to the primary and secondary objectives |
| assessments | include: |
| | Clinician Reported Outcomes (ClinRO) assessed by the investigator |
| | Investigator Global Assessment (IGA) |
| | Physician Assessment of Surface Area of Disease (PS AD) |
| | Reticulation, Erythema and Ulcerations score (REU) |
| | Lichen Planopilaris Activity Index (LPPAI) |
| | Patient reported outcomes (PRO) assessed by the subject: |
| | Patient Global Impression of Severity (PGIS) |
| | Patient Global Impression Change (PGIC) |
| | Patient assessment of itch |
| | Patient assessment of pain |
| | Oral Lichen Planus Symptom Severity Measure (OLPSSM) |
| | Dermatology Life Quality Index (DLQI) |
| | Epworth Sleepiness scale |
| | SCALPDEX |
| Pharmacokinetic | Pharmacokinetic assessments linked to exploratory objectives. |
| assessments | |
| Key safety | Evaluation of all AEs and SAEs |
| assessments | Physical examination |
| | Vital signs |
| | Laboratory evaluations (e.g. hematology, clinical chemistry) |
| | Immunogenicity (assessment of anti-secukinumab antibody |
| | development) |
| | Pregnancy |
| Other assessments | Exploratory biomarkers |
| | Immunogenicity |
| | Pharmacogenomic assessments |
| Data analysis | In each cohort, for secukinumab 300 mg and placebo treatment |
| | groups, the proportion of subjects classified as IGA responders after 16 |
| | weeks of treatment, will be modeled with a binomial distribution. A |
| | neutral, non-informative Beta (1/3, 1/3) distribution will be used as the |
| | prior for the response rate for secukinumab and placebo |
| | treatment groups. Based on the priors and the observed primary |
| | outcome, posterior distributions for the response rate in secukinumab |
| | and placebo treatment groups will be computed respectively for each |
| | cohort. The posterior distribution of the difference of response |
| | rates over placebo will be obtained by simulations. The posterior |
| | probabilities for a positive treatment difference will be assessed |
| | according to the dual efficacy criteria as a guide to decision making. In |
| | each cohort, secukinumab treatment will be assessed separately |
| | in comparison to the placebo group. Subjects who do not complete the |
| | 16-week treatment will be considered as non-responders. |

What is claimed is:

1. A method of treating lichen planopilaris (LPP), comprising subcutaneously (SC) administering to a patient in need thereof a dose of about 150 mg-about 300 mg of an Interleukin (IL)-17 antibody, or an antigen-binding fragment thereof, weekly during weeks 0, 1, 2, 3, and 4, and every four weeks thereafter, beginning during week 8, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

2. A method of treating lichen planus (LP), comprising subcutaneously (SC) administering to a patient in need thereof a dose of about 150 mg-about 300 mg of an Interleukin (IL)-17 antibody, or an antigen-binding fragment thereof, weekly during weeks 0, 1, 2, 3, and 4, and every two weeks thereafter, beginning during week 6, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

3. A method of treating lichen planus (LP), comprising intravenously (IV) administering to a patient in need thereof a dose of about 4 mg/kg-about 9 mg/kg (preferably about 6 mg/kg) of an Interleukin (IL)-17 antibody, or an antigen-binding fragment thereof, once during week 0, and thereafter administering an IV dose of about 2 mg/kg-about 4 mg/kg (preferably about 3 mg/kg) of the IL-17 antibody, or an antigen-binding fragment thereof every four weeks, beginning during week 4, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

4. The method according to any of claims 1-3, wherein the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE), and wherein the IL-17 antibody has an in vivo half-life of about 23 to about 30 days.

5. The method according to claim 1, wherein, if the patient does not adequately respond to treatment with the IL-17 antibody or antigen-binding fragment thereof following a period of every four week administration, then the IL-17 antibody or antigen-binding fragment thereof is administered to the patient every two weeks as a maintenance regimen.

6. The method according to any of claims 1-2, wherein the dose of IL-17 antibody or antigen-binding fragment thereof is 150 mg.

7. The method according to any of claims 1-2, wherein the dose of IL-17 antibody or antigen-binding fragment thereof is 300 mg.

8. The method according to any of the above claims, wherein prior to treatment with the IL-17 antibody or antigen-binding fragment thereof, the patient did not adequately respond to treatment with a lichenoid therapy selected from the group consisting of a topical therapy, a systemic therapy, phototherapy, a retinoid, and any combination thereof.

9. The method according to any of the above claims, wherein prior to treatment with the IL-17 antibody or antigen-binding fragment thereof, the patient was refractory to topical corticosteroid therapy or the patient did not adequately respond to treatment with a topical steroid.

10. The method according to any of the above claims, wherein during treatment with the IL-17 antibody or antigen-binding fragment thereof, the patient is concomitantly administered a lichenoid therapy selected from the group consisting of a topical therapy, a systemic therapy, phototherapy, a retinoid, and any combination thereof.

11. The method according to any of the above claims, wherein during treatment with the IL-17 antibody or antigen-binding fragment thereof, the patient is concomitantly administered at least one low to medium potency topical steroid.

12. The method according to any of claims 2-11, wherein the patient has biopsy-confirmed cutaneous lichen planus (CLP), biopsy-confirmed mucosal lichen planus (MLP) or biopsy-confirmed lichen planopilaris (LPP).

13. The method according to any of claims 2-12, wherein the patient has CLP and a baseline Body Surface Area (BSA) involvement of ≥3%, with or without nail involvement.

14. The method according to any of claims 2-12, wherein the patient has MLP and one or more affected locations selected from the oral cavity, genitals, and conjunctiva.

15. The method according to any of claims 1-12, wherein the patient has LPP and at least three active patches.

16. The method according to any of the above claims, wherein the patient:
a) has a baseline Investigator's Global Assessment (IGA) of ≥3; and
b) is refractory to topical corticosteroid therapy or has had an inadequate response to topical steroids.

17. The method according to claim 16, wherein following treatment with the IL-17 antibody or antigen-binding fragment thereof, the patient achieves at least two points improvement in IGA score versus baseline IGA score.

18. The method according to any of the above claims, wherein the patient is an adult.

19. The method according to any of the above claims, wherein the IL-17 antibody or antigen-binding fragment thereof is disposed in a pharmaceutical formulation, wherein said pharmaceutical formulation further comprises a buffer and a stabilizer.

20. The method according to claim 19, wherein the pharmaceutical formulation is in liquid form.

21. The method according to claim 19, wherein the pharmaceutical formulation is in lyophilized form.

22. The method according to any of claims 19-21, wherein the pharmaceutical formulation is disposed within at least one pre-filled syringe, at least one vial, at least one injection pen, or at least one autoinjector.

23. The method according to claim 22, wherein the at least one pre-filled syringe, at least one vial, at least one injection pen, or at least one autoinjector is disposed within a kit, and wherein said kit further comprises instructions for use.

24. The method according to any of claim 1-2 or 4-23, wherein the dose of the IL-17 antibody or antigen-binding fragment is 300 mg, which is administered to the patient as a single subcutaneous administration in a total volume of 2 milliliters (mL) from a formulation comprising 150 mg/ml of the IL-17 antibody or antigen-binding fragment, wherein the pharmacological exposure of the patient to the IL-17 antibody or antigen-binding fragment is equivalent to the pharmacological exposure of the patient to the IL-17 antibody or antigen-binding fragment using two separate subcutaneous administrations of a total volume of 1 ml each of the same formulation.

25. The method according to any of claim 1-2 or 4-23, wherein the dose of the IL-17 antibody or antigen-binding fragment administered to the patient is 300 mg, which is administered as two separate subcutaneous administrations in a volume of 1 mL each from a formulation comprising 150 mg/ml of the IL-17 antibody or antigen-binding fragment

26. The method according to any of the above claims, wherein the IL-17 antibody or antigen-binding fragment has a Tₘₐₓ of about 7-8 days.

27. The method according to any of the above claims, wherein the IL-17 antibody or antigen-binding fragment has an absolute bioavailability of about 60%-about 80%.

28. The method according to any of the above claims, wherein the IL-17 antibody or antigen-binding fragment is a human monoclonal antibody.

29. The method according to any of the above claims, wherein the IL-17 antibody or antigen-binding fragment is of the IgG₁/kappa isotype.

30. The method according to any of the above claims, wherein, when said method is used to treat a population of patients, at least 30% of said patients achieve an IGA 0/1 after 16 weeks of treatment.

31. The method according to any of the above claims, wherein, when said method is used to treat a population of patients, at least 40% of said patients achieve an IGA 0/1 after 16 weeks of treatment.

32. The method according to any of claim 2-12, 14, or 16-31, wherein the patient has MLP, and wherein the patient achieves an improvement in MLP as measured by the Modified Oral Mucositis Index (MOMI) following 16 weeks of treatment with the IL-17 antibody or antigen-binding fragment thereof.

33. The method according to any of the claim 1-12 or 15-31, wherein the patient achieves an improvement in LPP as measured by the Lichen Planopilaris Activity Index (LPPAI) following 16 weeks of treatment with the IL-17 antibody or antigen-binding fragment thereof.

34. The method according to any of the above claims, wherein the patient achieves an improvement in pruritus as measured by the Peak Pruritus Numerical Rating Scale (NRS) following 16 weeks of treatment with the IL-17 antibody or antigen-binding fragment thereof.

35. The method according to any of the above claims, wherein the patient achieves an improvement in pain as measured by a Visual Analogue Scale (VAS) following 16 weeks of treatment with the IL-17 antibody or antigen-binding fragment thereof.

36. The method according to any of the above claims, wherein the patient achieves an improvement in quality of life as measured by the Dermatology Life Quality Index (DLQI) 0/1 following 16 weeks of treatment with the IL-17 antibody or antigen-binding fragment thereof.

37. The method according to any of the above claims, wherein the patient has MLP, and wherein the patient achieves an improvement in MLP as measured the Oral Health Impact Profile (OHIP-14) following 16 weeks of treatment with the IL-17 antibody or antigen-binding fragment thereof.

38. The method according to any of the above claims, wherein the patient is treated with the IL-17 antibody or antigen-binding fragment thereof for at least one year.

39. The method according to any of the above claims, wherein the IL-17 antibody or antigen-binding fragment is secukinumab.

40. A method of treating an adult patient with lichen planopilaris (LPP) that is inadequately controlled with topical corticosteroid therapy or for whom topical corticosteroid therapy is not advisable, comprising administering a dose of about 300 mg secukinumab subcutaneously to said patient during week 0, 1, 2, 3, and 4, and then every four weeks thereafter.

41. A method of treating an adult patient with lichen planus (LP) inadequately controlled with topical corticosteroid therapy or for whom topical corticosteroid therapy is not advisable, comprising administering a dose of about 300 mg secukinumab subcutaneously to said patient during week 0, 1, 2, 3, and 4, and then every two weeks thereafter.

42. A method of treating an adult patient with lichen planus (LP) inadequately controlled with topical corticosteroid therapy or for whom topical corticosteroid therapy is not advisable, comprising, intravenously (IV) administering to the patient a dose of about 6 mg/kg secukinumab once during week 0, and thereafter administering an IV dose of about 3 mg/kg secukinumab every four weeks, beginning during week 4.

43. The method of either claim 41 or 42, wherein said patient has cutaneous lichen planus (CLP), mucosal lichen planus (MLP) or lichen planopilaris (LPP).

44. The method of either claim 41 or 42, wherein the CLP, MLP or LPP is biopsy-confirmed.


SEQUENCE LISTING (21 sequences)

SEQ ID NO: 1 - protein, 5 aa, artificial
  CDR1 = hypervariable region 1 of heavy chain of AIN457
NYWMN

SEQ ID NO: 2 - protein, 17 aa, ARTIFICIAL
  CDR2 = hypervariable region 2 of heavy chain of AIN457
AINQDGSEKYYVGSVKG

SEQ ID NO: 3 - protein, 18 aa, ARTIFICIAL
  CDR3 = hypervariable region 3 of heavy chain of AIN457
DYYDILTDYYIHYWYFDL

SEQ ID NO: 4 - protein, 12 aa, ARTIFICIAL
  CDR1′ = hypervariable region 1 of light chain of AIN457
RASQSVSSSYLA

SEQ ID NO: 5 - protein, 7 aa, ARTIFICIAL
  CDR2′ = hypervariable region 2 of light chain AIN457
GASSRAT

SEQ ID NO: 6 - protein, 9 aa, ARTIFICIAL
  CDR3′ = hypervariable region 3 of light chain AIN457
QQYGSSPCT

SEQ ID NO: 7 - DNA, 381 bp, HOMO SAPIENS
  CDS (1)..(381)
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, HOMO SAPIENS [sequence omitted]

SEQ ID NO: 9 - DNA, 327 bp, HOMO SAPIENS
  CDS (1)..(327)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, HOMO SAPIENS
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, artificial
  CDR1-x = hypervariable domain x of heavy chain of AIN457
GFTFSNYWMN

SEQ ID NO: 12 - protein, 11 aa, artificial
  CDR2-x = hypervariable domain of heavy chain x of AIN457
AINQDGSEKYY

SEQ ID NO: 13 - protein, 23 aa, ARTIFICIAL
  CDR3-x = hypervariable domain x of heavy chain AIN457
CVRDYYDILTDYYIHYWYFDLWG

SEQ ID NO: 14 - protein, 215 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 457 aa, homo sapiens
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 7 aa, artificial
  IMGT LCDR1
QSVSSSY

SEQ ID NO: 17 - protein, 3 aa, artificial
  IMGT LCDR2
GAS

SEQ ID NO: 18 - protein, 9 aa, Artificial
  IMGT LCDR3
QQYGSSPCT

SEQ ID NO: 19 - protein, 8 aa, artificial
  IMGT HCDR1
GFTFSNYW

SEQ ID NO: 20 - protein, 8 aa, Artificial
  IMGT HCDR2
INQDGSEK

SEQ ID NO: 21 - protein, 20 aa, Artificial [sequence omitted]